(19) United States
(12) Patent Application Publication
(10) Pub. No.: US 2026/0071013 A1
(43) Pub. Date: Mar. 12, 2026

(54) COMPOSITION CONTAINING FLUORINE-CONTAINING POLYMER, METHOD FOR PRODUCING FLUORINE-CONTAINING POLYMER, AND ULTRAVIOLET LIGHT-EMITTING ELEMENT LENS CONTAINING FLUORINE-CONTAINING POLYMER

(71) Applicant: DAIKIN INDUSTRIES, LTD., Osaka (JP)

(72) Inventors: Daichi MARI, Osaka (JP); Yuki SUZUKI, Osaka (JP); Atsushi SHIRAI, Osaka (JP); Noriyuki SHINOKI, Osaka (JP); Michiaki OKADA, Osaka (JP); Yoshichika KUROKI, Osaka (JP)

(73) Assignee: DAIKIN INDUSTRIES, LTD., Osaka (JP)

(21) Appl. No.: 19/392,415

(22) Filed: Nov. 18, 2025

Related U.S. Application Data
(63) Continuation of application No. PCT/JP2024/018035, filed on May 15, 2024.

(30) Foreign Application Priority Data
May 22, 2023 (JP) 2023-083945

(51) Int. Cl. C08F 114/22; C08F 2/06; C08F 6/12; C08K 5/14; G02B 1/04

(52) U.S. Cl. CPC C08F 114/22; C08F 2/06; C08F 6/12; C08K 5/14; G02B 1/041

(57) ABSTRACT

An object of the present disclosure is to provide a composition comprising a fluorine-containing polymer with high ultraviolet-light permeability, a method for producing a fluorine-containing polymer with high ultraviolet-light permeability, a lens for ultraviolet-light-emitting elements comprising a fluorine-containing polymer with high ultraviolet-light permeability, and the like. Specifically, provided is a composition comprising a fluorine-containing polymer (A), and at least one compound (BC) selected from the group consisting of a polymerization initiator (B) and a decomposed product (C) of the polymerization initiator (B), wherein the compound (BC) is contained in an amount of 0.1 to 100 ppm by mass based on the mass of the composition, and the fluorine-containing polymer (A) comprises as a main component a structural unit represented by formula (A):

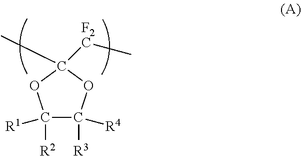

wherein R¹ to R⁴ each independently represent a fluorine atom, a C1-C5 perfluoroalkyl group, or a C1-C5 perfluoroalkoxy group.

### TECHNICAL FIELD

The present disclosure relates to a composition comprising a fluorine-containing polymer, a method for producing a fluorine-containing polymer, and a lens for ultraviolet-light-emitting elements comprising a fluorine-containing polymer.

### BACKGROUND ART

Fluorine-containing polymers containing an oxolane ring are known to exhibit high transparency, and applications in the field of optics are under consideration. Meanwhile, the use of ultraviolet-light-emitting elements has grown in recent years, and there is a demand for polymers with high ultraviolet-light permeability.

PTL 1 and PTL 2 disclose a fluorine-containing polymer that does not contain a carbonyl group at its terminal position, thereby suppressing the yellowing of the fluorine-containing polymer and enhancing the ultraviolet light permeability.

### CITATION LIST

### Patent Literature

PTL 1: JP2021-155736A
PTL 2: WO2014/156996

### SUMMARY

The present disclosure typically encompasses the following embodiments.

A composition comprising:
a fluorine-containing polymer (A); and
at least one compound (BC) selected from the group consisting of a polymerization initiator (B) and a decomposed product (C) of the polymerization initiator (B),
wherein the compound (BC) is contained in an amount of 0.1 to 100 ppm by mass based on the mass of the composition, and
the fluorine-containing polymer (A) comprises as a main component a structural unit represented by formula (A):

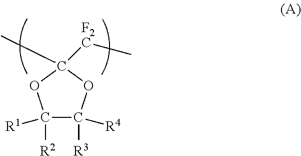

wherein R¹ to R⁴ each independently represent a fluorine atom, a C1-C5 perfluoroalkyl group, or a C1-C5 perfluoroalkoxy group.

### Advantageous Effects

The present disclosure provides a composition comprising a fluorine-containing polymer with high ultraviolet-light permeability, a method for producing a fluorine-containing polymer with high ultraviolet-light permeability, a lens for ultraviolet-light-emitting elements comprising a fluorine-containing polymer with high ultraviolet-light permeability, and the like.

The production method of the present disclosure enables the production of a fluorine-containing polymer with high ultraviolet-light permeability through a polymerization step and filtration step without requiring a step of removing the polymerization initiator and/or a decomposed product thereof after polymerization.

### DESCRIPTION OF EMBODIMENTS

The above overview of the present disclosure is not intended to describe each of the disclosed embodiments or all of the implementations of the present disclosure.

The following description of the present disclosure more specifically exemplifies illustrative embodiments.

Guidance is provided through examples in several parts of the present disclosure, and these examples can be used in various combinations.

In each case, the group of examples can function as a non-exclusive and representative group.

All publications, patents, and patent applications cited herein are incorporated herein by reference in their entirety.

### Terms

Unless otherwise specified, the symbols and abbreviations in the present specification can be understood in the context of the present specification in the meanings commonly used in the technical field to which the present disclosure belongs.

In the present specification, the terms “comprise” and “contain” are used with the intention of including the terms “consisting essentially of” and “consisting of.”

Unless otherwise specified, the steps, treatments, or operations described in the present specification may be performed at room temperature.

In the present specification, “room temperature” can refer to a temperature within the range of 10 to 40° C.

In the present specification, the phrase “Cn-Cm” (n and m are each a number) indicates that the number of carbon atoms is n or more and m or less, as a person skilled in the art would generally understand.

In the present specification, unless otherwise specified, the “alkyl group” refers to a linear, branched, or cyclic alkyl group. Examples of alkyl groups include linear or branched C1-C20 alkyl groups (for example, C1-C10, preferably C1-C7, more preferably C1-C6, still more preferably C1-C4, and particularly preferably C1-C3 alkyl groups), such as methyl, ethyl, propyl (e.g., n-propyl, isopropyl), butyl (e.g., n-butyl, isobutyl, sec-butyl, tert-butyl), pentyl (e.g., n-pentyl, tert-pentyl, neopentyl, isopentyl, sec-pentyl, 3-pentyl), hexyl, heptyl, octyl, nonyl, decyl, undecyl, dodecyl, tridecyl, tetradecyl, pentadecyl, hexadecyl, heptadecyl, octadecyl, nonadecyl, and icosyl; and cyclic C3-C10 alkyl groups (for example, C3-C6, C4-C6, C3-C5, C5-C6, or C4-C8 alkyl groups), such as cyclopropyl, cyclobutyl, cyclopentyl, cyclohexyl, cycloheptyl, cyclooctyl, and adamantyl.

In the present specification, unless otherwise specified, the “alkoxy group” refers to a linear, branched, or cyclic alkoxy group. The alkoxy group may be represented by RO—, wherein R represents an alkyl group (e.g., a C1-C20 alkyl group, C1-C10 alkyl group, C1-C7 alkyl group, C1-C6 alkyl group, C1-C4 alkyl group, or C1-C3 alkyl group).

Examples of alkoxy groups include linear or branched C1-C20 alkoxy groups (e.g., C1-C10, preferably C1-C7, more preferably C1-C6, still more preferably C1-C4, and particularly preferably C1-C3 alkoxy groups), such as methoxy, ethoxy, propoxy (e.g., n-propoxy, isopropoxy), butoxy (e.g., n-butoxy, isobutoxy, sec-butoxy, tert-butoxy), pentyloxy (e.g., n-pentyloxy, tert-pentyloxy, neopentyloxy, isopentyloxy, sec-pentyloxy, 3-pentyloxy), hexyloxy, heptyloxy, octyloxy, nonyloxy, decyloxy, undecyloxy, dodecyloxy, tridecyloxy, tetradecyloxy, pentadecyloxy, hexadecyloxy, heptadecyloxy, octadecyloxy, nonadecyloxy, and icosyloxy; and cyclic C3-C10 alkoxy groups (for example, C3-C6, C4-C6, C3-C5, C5-C6, and C4-C8 alkoxy groups), such as cyclopropoxy, cyclobutoxy, cyclopentyloxy, cyclohexyloxy, cycloheptyloxy, cyclooctyloxy, and adamantyloxy.

In the present specification, unless otherwise specified, the “fluoroalkyl group” refers to a linear, branched, or cyclic alkyl group in which at least one hydrogen atom is replaced by a fluorine atom. The fluoroalkyl group also includes perfluoroalkyl groups, in which all hydrogen atoms in the alkyl are replaced by fluorine atoms.

The number of carbon atoms in the fluoroalkyl group may be, for example, 1 to 20, 1 to 10, 1 to 7, 1 to 6, 1 to 5, 1 to 4, 1 to 3, 1 to 2, or 1.

Examples of fluoroalkyl groups include linear or branched C1-C20 fluoroalkyl groups (for example, C1-C10, C1-C4, or C1-C3, preferably C1-C7, and more preferably C1-C6 fluoroalkyl group) (preferably perfluoroalkyl groups), such as methyl having 1 to 3 fluorine atoms, ethyl having 1 to 5 fluorine atoms, propyl (such as n-propyl and isopropyl) having 1 to 7 fluorine atoms, butyl (such as n-butyl, isobutyl, sec-butyl, and tert-butyl) having 1 to 9 fluorine atoms, pentyl (such as n-pentyl, tert-pentyl, neopentyl, isopentyl, sec-pentyl, and 3-pentyl) having 1 to 11 fluorine atoms, hexyl having 1 to 13 fluorine atoms, heptyl having 1 to 15 fluorine atoms, octyl having 1 to 17 fluorine atoms, nonyl having 1 to 19 fluorine atoms, decyl having 1 to 21 fluorine atoms, undecyl having 1 to 23 fluorine atoms, dodecyl having 1 to 25 fluorine atoms, tridecyl having 1 to 27 fluorine atoms, tetradecyl having 1 to 29 fluorine atoms, pentadecyl having 1 to 31 fluorine atoms, hexadecyl having 1 to 33 fluorine atoms, heptadecyl having 1 to 35 fluorine atoms, octadecyl having 1 to 37 fluorine atoms, nonadecyl having 1 to 39 fluorine atoms, and icosyl having 1 to 41 fluorine atoms; and cyclic C3-C10 fluoroalkyl groups (for example, C3-C6, C4-C6, C3-C5, C5-C6, and C4-C8 fluoroalkyl groups) (preferably perfluoroalkyl groups), such as cyclofluoropropyl, cyclofluorobutyl, cyclofluoropentyl, cyclofluorohexyl, cyclofluoroheptyl, cyclofluorooctyl, and fluoroadamantyl.

The number of fluorine atoms in the fluoroalkyl group may be one to the maximum substitutable number, such as 1 to 41, 1 to 21, 1 to 19, 1 to 17, 1 to 15, 1 to 13, 1 to 11, 1 to 9, 1 to 7, 1 to 5, or 1 to 3.

Specific examples of the “fluoroalkyl group” include linear or branched fluoroalkyl groups, such as monofluoromethyl, difluoromethyl, trifluoromethyl (CF₃—), 2,2,2-trifluoroethyl, perfluoroethyl group (C₂F₅—), tetrafluoropropyl group (e.g., HCF₂CF₂CH₂—), hexafluoropropyl (e.g., (CF₃)₂CH—), perfluoropropyl (e.g., CF₃CF₂CF₂—, (CF₃)₂CF—), perfluorobutyl (e.g., CF₃CF₂CF₂CF₂—, (CF₃)₂CFCF₂—, (CF₃)₃—), decafluorohexyl (e.g., HCF₂CF₂CF₂CF₂CF₂—), octafluoropentyl (e.g., HCF₂CF₂CF₂CF₂CH₂—), perfluoropentyl (e.g., CF₃CF₂CF₂CF₂CF₂—, (CF₃)₂CFCF₂CF₂—), perfluorohexyl (e.g., CF₃CF₂CF₂CF₂CF₂CF₂—, (CF₃)₂CFCF₂CF₂CF₂—), perfluoroheptyl (e.g., CF₃CF₂CF₂CF₂CF₂CF₂CF₂—, (CF₃)₂CFCF₂CF₂CF₂CF₂—), and perfluorohexadecyl (e.g., CF₃CF₂CF₂CF₂CF₂CF₂CF₂CF₂CF₂CF₂CF₂CF₂CF₂CF₂CF₂CF₂—); cyclic C3-C10 fluoroalkyl groups (preferably perfluoroalkyl groups), such as cyclofluoropropyl, cyclofluorobutyl, cyclofluoropentyl, cyclofluorohexyl, cyclofluoroheptyl, cyclofluorooctyl, and fluoroadamantyl.

In the present specification, unless otherwise specified, the “fluoroalkoxy group” refers to a linear, branched, or cyclic alkoxy group in which at least one hydrogen atom is replaced by a fluorine atom. The fluoroalkoxy group also includes perfluoroalkoxy groups, in which all hydrogen atoms in the alkoxy are replaced by fluorine atoms.

The number of carbon atoms in the fluoroalkoxy group may be, for example, 1 to 20, 1 to 10, 1 to 7, 1 to 6, 1 to 5, 1 to 4, 1 to 3, 1 to 2, or 1.

Examples of fluoroalkoxy groups include linear or branched C1-C20 fluoroalkoxy groups (for example, C1-C10, preferably C1-C7, more preferably C1-C6, still more preferably C1-C4, and particularly preferably C1-C3 fluoroalkoxy groups) (preferably perfluoroalkoxy groups), such as methoxy having 1 to 3 fluorine atoms, ethoxy having 1 to 5 fluorine atoms, propoxy (e.g., n-propoxy, isopropoxy) having 1 to 7 fluorine atoms, butoxy (e.g., n-butoxy, isobutoxy, sec-butoxy, tert-butoxy) having 1 to 9 fluorine atoms, pentyloxy (e.g., n-pentyloxy, tert-pentyloxy, neopentyloxy, isopentyloxy, sec-pentyloxy, 3-pentyloxy) having 1 to 11 fluorine atoms, hexyloxy having 1 to 13 fluorine atoms, heptyloxy having 1 to 15 fluorine atoms, octyloxy having 1 to 17 fluorine atoms, nonyloxy having 1 to 19 fluorine atoms, decyloxy having 1 to 21 fluorine atoms, undecyloxy having 1 to 23 fluorine atoms, dodecyloxy having 1 to 25 fluorine atoms, tridecyloxy having 1 to 27 fluorine atoms, tetradecyloxy having 1 to 29 fluorine atoms, pentadecyloxy having 1 to 31 fluorine atoms, hexadecyloxy having 1 to 33 fluorine atoms, heptadecyloxy having 1 to 35 fluorine atoms, octadecyloxy having 1 to 37 fluorine atoms, nonadecyloxy having 1 to 39 fluorine atoms, and icosyloxy having 1 to 41 fluorine atoms; and cyclic C3-C10 fluoroalkoxy groups (for example, C3-C6, C4-C6, C3-C5, C5-C6, and C4-C8 fluoroalkoxy groups), such as cyclofluoropropoxy, cyclofluorobutoxy, cyclofluoropentyloxy, cyclofluorohexyloxy, cyclofluoroheptyloxy, cyclofluorooctyloxy, and fluoroadamantyloxy.

The number of fluorine atoms in the fluoroalkoxy group may be one to the maximum substitutable number, such as 1 to 41, 1 to 21, 1 to 19, 1 to 17, 1 to 15, 1 to 13, 1 to 11, 1 to 9, 1 to 7, 1 to 5, or 1 to 3.

Specific examples of the “fluoroalkoxy group” include linear or branched fluoroalkoxy groups, such as monofluoromethoxy, difluoromethoxy, trifluoromethoxy (CF₃O—), 2,2,2-trifluoroethoxy, perfluoroethoxy (C₂F₅O—), tetrafluoropropoxy (e.g., HCF₂CF₂CH₂O—), hexafluoropropoxy (e.g., (CF₃)₂CHO—), perfluoropropoxy (e.g., CF₃CF₂CF₂O—, (CF₃)₂CFO—), perfluorobutoxy (e.g., CF₃CF₂CF₂CF₂O—, (CF₃)₂CFCF₂O—, (CF₃)₃O—), decafluorohexyl oxy (e.g., HCF₂CF₂CF₂CF₂CF₂O—), octafluoropentyloxy (e.g., HCF₂CF₂CF₂CF₂CH₂O—), perfluoropentyloxy (e.g., CF₃CF₂CF₂CF₂CF₂O—, (CF₃)₂CFCF₂CF₂O—), perfluorohexyloxy (e.g., CF₃CF₂CF₂CF₂CF₂CF₂O—, (CF₃)₂CFCF₂CF₂CF₂O—), perfluoroheptyloxy (e.g., CF₃CF₂CF₂CF₂CF₂CF₂CF₂O—, (CF₃)₂CFCF₂CF₂CF₂CF₂O—), and perfluorohexadecyloxy (e.g., CF₃CF₂CF₂CF₂CF₂CF₂CF₂CF₂CF₂CF₂CF₂CF₂CF₂CF₂CF₂CF₂O—); and cyclic C3-C10 fluoroalkoxy groups (preferably perfluoroalkoxy groups), such as cyclofluoropropoxy, cyclofluorobutoxy, cyclofluoropentyloxy, cyclofluorohexyloxy, cyclofluoroheptyloxy, cyclofluorooctyloxy, and fluoroadamantyloxy.

In the present specification, unless otherwise specified, the “4- to 20-membered ring comprising 4 to 20 carbon atoms as ring-constituting atoms” includes cycloalkane rings, such as a cyclopropane ring, cyclobutane ring, cyclopentane ring, cyclohexane ring, cycloheptane ring, cyclooctane ring, cyclononane ring, cyclodecane ring, cycloundecane ring, and cyclododecane ring (preferably cyclobutane ring, cyclopentane ring, cyclohexane ring); non-aromatic unsaturated hydrocarbon rings, such as cyclobutadiene ring and cyclooctatetraene ring; aromatic rings, such as benzene ring, naphthalene ring, anthracene ring, and phenanthrene ring; and the like.

In the present specification, unless otherwise specified, examples of the “4- to 22-membered ring comprising 3 to 20 carbon atoms and one or two oxygen atoms or one sulfur atom as ring-constituting atoms” include oxygen-containing saturated heterocyclic rings having one oxygen atom as a ring-constituting atom, such as an oxetane ring, tetrahydrofuran ring, tetrahydropyran ring, oxepane ring, oxocane ring, and oxonane ring; oxygen-containing unsaturated heterocyclic rings having one oxygen atom as a ring-constituting atom, such as an oxete ring, furan ring, pyran ring, oxepine ring, oxocine ring, and oxonine ring; oxygen-containing saturated heterocyclic rings having two oxygen atoms as ring-constituting atoms, such as a dioxetane ring, dioxolane ring, dioxane ring, dioxepane ring, dioxocane ring; oxygen-containing unsaturated heterocyclic rings having two oxygen atoms as ring-constituting atoms, such as a dioxete ring, dioxolene ring, dihydrodioxine ring, dihydrodioxepin ring, and tetrahydrodioxocine ring; sulfur-containing unsaturated heterocyclic rings, such as a thiete ring, thiophene ring, thiopyran ring, thiepine ring, thiocine ring, and thionine ring; sulfur-containing saturated heterocyclic rings, such as a thietane ring, tetrahydrothiophene ring, thiane ring, thiepane ring, thiocane ring, and thionane ring; and the like.

In the present specification, unless otherwise specified, the “4- to 20-membered ring comprising 4 to 20 carbon atoms as ring-constituting atoms and at least one fluorine atom” refers to a ring in which at least one hydrogen atom bonded to a ring-constituting atom of a 4- to 20-membered ring comprising 4 to 20 carbon atoms as ring-constituting atoms is replaced by a fluorine atom. The number of the fluorine atoms may be one to the maximum substitutable number. Preferably, the ring is a ring in which all hydrogen atoms bonded to the ring-constituting atoms are replaced by fluorine atoms (i.e., a perfluoro ring).

In the present specification, unless otherwise specified, the “4- to 22-membered ring comprising 3 to 20 carbon atoms and one or two oxygen atoms or one sulfur atom as ring-constituting atoms and at least one fluorine atom” refers to a ring in which at least one hydrogen atom bonded to a ring-constituting atom of a 4- to 22-membered ring comprising 3 to 20 carbon atoms and one or two oxygen atoms or one sulfur atom as ring-constituting atoms is replaced by a fluorine atom. The number of the fluorine atoms may be one to the maximum substitutable number. Preferably, the ring is a ring in which all hydrogen atoms bonded to the ring-constituting atoms are replaced by fluorine atoms (i.e., a perfluoro ring).

In the present specification, unless otherwise specified, the “aryl group” may refer to a monocyclic, bicyclic, or tricyclic aryl. The aryl group may be a C6-C14 aryl group, C6-C10 aryl group, C6-C9 aryl group, or C6 aryl group. Examples of aryl groups include phenyl, 1-naphthyl, 2-naphthyl, indenyl, biphenylyl, anthryl, phenanthryl, and the like. The aryl group may have methyl, ethyl, propyl, and the like, which may be identical or different when present in multiple. The number of groups that the aryl group may have may be six, five, four, three, two, one, or zero.

In the present specification, unless otherwise specified, the “aryl group having a fluorine atom” refers to an aryl group in which at least one hydrogen atom is replaced by a fluorine atom, and also includes perfluoroaryl groups, in which all hydrogen atoms in the aryl are replaced by fluorine atoms. Preferred are perfluoroaryl groups.

The number of fluorine atoms in the aryl group may be one to the maximum substitutable number, such as 1 to 16, 1 to 15, 1 to 14, 1 to 13, 1 to 12, 1 to 11, 1 to 10, 1 to 9, 1 to 5, or 1 to 3.

Examples of aryl groups having a fluorine atom include phenyl having a fluorine atom, 1-naphthyl having a fluorine atom, 2-naphthyl having a fluorine atom, indenyl having a fluorine atom, biphenylyl having a fluorine atom, anthryl having a fluorine atom, phenanthryl having a fluorine atom, and the like.

In the present specification, unless otherwise specified, examples of alkali metals include lithium, sodium, potassium, rubidium, cesium, and francium. Preferred examples include lithium, sodium, and potassium, and more preferred examples include sodium and potassium.

In the present specification, unless otherwise specified, examples of alkaline earth metal include beryllium, magnesium, calcium, strontium, barium, and radium. Preferred examples include magnesium and calcium.

### Method for Producing Fluorine-Containing Polymer (A)

One embodiment of the present disclosure includes a method for producing a fluorine-containing polymer (A). The production method of the present disclosure enables the production of a fluorine-containing polymer with high ultraviolet-light permeability. The production method of the present disclosure comprises a polymerization step and a filtration step, and the production method may comprise or may not comprise a step of removing the polymerization initiator and/or a decomposed product thereof. Therefore, the production method of the present disclosure is simple and easy.

The production method of the present disclosure comprises a polymerization step of polymerizing a fluorine-containing monomer (M) represented by formula (M):

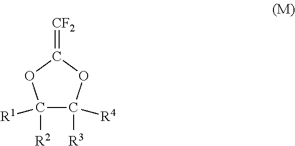

wherein R¹ to R⁴ each independently represent a fluorine atom, a C1-C5 perfluoroalkyl group, or a C1-C5 perfluoroalkoxy group in the presence of the fluorine-containing monomer (M), a polymerization initiator (B), and a solvent (D) to produce a fluorine-containing polymer (A) as a precipitate.

The fluorine-containing polymer (A) comprises as a main component a structural unit represented by formula (A):

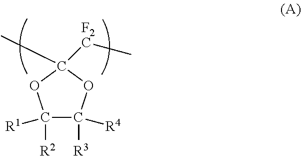

wherein R¹ to R⁴ are as defined above (which may be referred to as “the structural unit (A)” in the present specification).

A value (R_{A}) of a solubility index R, calculated based on the Hansen solubility parameters of the solvent (D) and the Hansen solubility parameters of the fluorine-containing polymer (A), may be 4 or more.

A value (R_{B}) of a solubility index R, calculated based on the Hansen solubility parameters of the solvent (D) and the Hansen solubility parameters of the polymerization initiator (B), may be 9 or less when the mass percentage of fluorine atoms in the polymerization initiator (B) is less than 50 mass % and may be 6 or less when the same mass percentage is 50 mass % or more.

A value (R_{C}) of a solubility index R, calculated based on the Hansen solubility parameters of the solvent (D) and the Hansen solubility parameters of the decomposed product (C) of the polymerization initiator (B), may be 9 or less when the mass percentage of fluorine atoms in the decomposed product (C) is less than 50 mass % and may be 6 or less when the same mass percentage is 50 mass % or more.

The solubility index R is calculated according to the following equation:

$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$

wherein δD₁, δP₁, and δH₁ respectively represent the dispersion parameter, the polarity parameter, and the hydrogen bonding parameter in the Hansen solubility parameters of element 1, and δD₂, δP₂, and δH₂ respectively represent the dispersion parameter, the polarity parameter, and the hydrogen bonding parameter in the Hansen solubility parameters of element 2.

### Fluorine-Containing Polymer (A)

The fluorine-containing polymer (A) comprises as a main component a structural unit represented by formula (A):

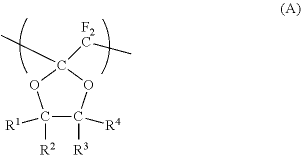

wherein R¹ to R⁴ each independently represent a fluorine atom, a C1-C5 perfluoroalkyl group, or a C1-C5 perfluoroalkoxy group.

The phrase “comprises as a main component a structural unit” means that the proportion of this structural unit in all of the structural units in the fluorine-containing polymer (A) is 50 mol % or more.

The proportion of the structural unit (A) in the fluorine-containing polymer (A) is preferably 80 mol % or more, more preferably 90 mol % or more, and particularly preferably 100 mol %.

The structural unit (A) may be one or more types, preferably one to three types, more preferably one or two types, and particularly preferably one type.

In the structural unit (A), R¹ to R⁴ may each independently represent a fluorine atom, a C1-C5 linear or branched perfluoroalkyl group, or a C1-C5 linear or branched perfluoroalkoxy group.

In the structural unit (A), R¹ to R⁴ may each independently represent a fluorine atom, a C1-C4 linear or branched perfluoroalkyl group, or a C1-C4 linear or branched perfluoroalkoxy group.

In the structural unit (A), R¹ to R⁴ may each independently represent a fluorine atom, a C1-C3 linear or branched perfluoroalkyl group, or a C1-C3 linear or branched perfluoroalkoxy group.

In the structural unit (A), it is preferable that R¹ to R⁴ each independently represent a fluorine atom, trifluoromethyl, perfluoroethyl, or trifluoromethoxy.

In the structural unit (A), it is more preferable that R¹ to R⁴ each independently represent a fluorine atom, trifluoromethyl, or trifluoromethoxy.

At least one of the groups R¹ to R⁴ may represent a fluorine atom, and the remaining groups may, if multiple, independently represent a C1-C2 perfluoroalkyl group or a C1-C2 perfluoroalkoxy group.

At least two of the groups R¹ to R⁴ may each represent a fluorine atom, and the remaining groups may, if multiple, independently represent a C1-C2 perfluoroalkyl group or a C1-C2 perfluoroalkoxy group.

At least three of the groups R¹ to R⁴ may each represent a fluorine atom, and the remaining group may represent a C1-C2 perfluoroalkyl group or a C1-C2 perfluoroalkoxy group.

At least three of the groups R¹ to R⁴ may each represent a fluorine atom, and the remaining group may represent a C1-C2 perfluoroalkyl group.

R¹ to R⁴ may all represent fluorine atoms.

The structural unit (A) may be a structural unit represented by the following formula (A1) (which may be referred to as “the structural unit (A1)” in the present specification). In the present specification, a fluorine-containing polymer (A) comprising the structural unit (A1) as the main component may be referred to as “the fluorine-containing polymer (A1).”

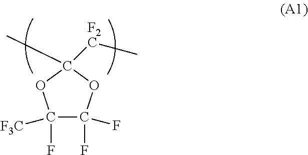

The C1-C5 perfluoroalkyl group represented by each of R¹ to R⁴ may be, for example, a linear or branched C1-C5 fluoroalkyl group, a linear or branched C1-C4 fluoroalkyl group, a linear or branched C1-C3 fluoroalkyl group, or a C1-C2 fluoroalkyl group.

The linear or branched C1-C5 fluoroalkyl group is preferably a linear or branched C1-C5 perfluoroalkyl group.

The linear or branched C1-C4 fluoroalkyl group is preferably a linear or branched C1-C4 perfluoroalkyl group.

The linear or branched C1-C3 fluoroalkyl group is preferably a linear or branched C1-C3 perfluoroalkyl group.

The C1-C2 fluoroalkyl group is preferably a C1-C2 perfluoroalkyl group.

The fluorine-containing polymer (A) may comprise a structural unit derived from a fluoroolefin (which may be referred to as “the fluoroolefin unit” in the present specification), in addition to the structural unit (A) contained as the main component. As the fluoroolefin unit, a single type may be used singly, or two or more types may be used in combination. The proportion of the fluoroolefin unit in all of the structural units in the fluorine-containing polymer (A) may be set at 50 mol % or less, preferably 20 mol % or less, more preferably 10 mol % or less, and particularly preferably 0 mol %.

The fluoroolefin unit is a monomer unit formed after polymerization of a monomer containing one or more fluorine atoms and one or more carbon-carbon double bonds.

The atoms constituting the fluoroolefin unit may be only fluorine atoms, halogen atoms other than fluorine atoms, carbon atoms, hydrogen atoms, and oxygen atoms.

The atoms constituting the fluoroolefin unit may be only fluorine atoms, halogen atoms other than fluorine atoms, carbon atoms, and hydrogen atoms.

The atoms constituting the fluoroolefin unit may be only fluorine atoms, carbon atoms, and hydrogen atoms.

The atoms constituting the fluoroolefin unit may be only fluorine atoms and carbon atoms.

The fluoroolefin unit includes a fluorine-containing perhaloolefin unit, a vinylidene fluoride unit (—CH₂—CF₂—), a trifluoroethylene unit (—CFH—CF₂—), a pentafluoropropylene unit (—CFH—CF(CF₃)—,—CF₂—CF(CHF₂)—), and a 1,1,1,2-tetrafluoro-2-propylene unit (—CH₂—CF(CF₃)—).

The fluorine-containing perhaloolefin unit is a structural unit formed after polymerization of a monomer that contains one or more fluorine atoms and one or more carbon-carbon double bonds with one or more optional halogen atoms other than fluorine atoms.

The fluorine-containing perhaloolefin unit includes a chlorotrifluoroethylene unit (—CFCl—CF₂—), a tetrafluoroethylene unit (—CF₂—CF₂—), a hexafluoropropylene unit (—CF₂—CF(CF₃)—), a perfluoro(methyl vinyl ether) unit (—CF₂—CF(OCF₃)—), a perfluoro(ethyl vinyl ether) unit (—CF₂—CF(OC₂F₅)—), a perfluoro (propyl vinyl ether) unit (—CF₂—CF(OCF₂C₂F₅)—), a perfluoro(butyl vinyl ether) unit (—CF₂—CF(O(CF₂)₂C₂F₅)—), and a perfluoro(2,2-dimethyl-1,3-dioxol) unit (—CF-CAF— wherein A represents a perfluorodioxolane ring formed together with an adjacent carbon atom in the formula with two trifluoromethyl groups bound to the carbon atom at the 2-position of the dioxolane ring).

The fluoroolefin unit includes a chlorotrifluoroethylene unit, a tetrafluoroethylene unit, a hexafluoropropylene unit, a perfluoro(methyl vinyl ether) unit, and a perfluoro(propyl vinyl ether) unit.

The fluorine-containing polymer (A) may further contain one or more other structural units in addition to the structural unit (A) and the fluoroolefin unit; however, it is preferable that the fluorine-containing polymer (A) does not contain other structural units.

Such other structural units include CH₂═CHRf (wherein Rf represents a C1-C10 fluoroalkyl group) units, alkyl vinyl ether units (e.g., a cyclohexyl vinyl ether unit, ethyl vinyl ether unit, butyl vinyl ether unit, and methyl vinyl ether unit), alkenyl vinyl ether units (e.g., a polyoxyethylene allyl ether unit and ethyl allyl ether unit), organosilicon compound units having a reactive α,β-unsaturated group (e.g., a vinyltrimethoxysilane unit, vinyltriethoxysilane unit, and a vinyltris(methoxyethoxy)silane unit), acrylic ester units (e.g., a methyl acrylate unit and ethyl acrylate unit), methacrylic ester units (e.g., a methyl methacrylate unit and ethyl methacrylate unit), vinyl ester units (e.g., a vinyl acetate unit, vinyl benzoate unit, and a VeoVA (vinyl ester produced by Shell plc) unit), and the like.

The proportion of the other structural units in all of the structural units may be, for example, 0 to 20 mol %, 0 to 10 mol %, or the like.

The mass average molecular weight of the fluorine-containing polymer (A) may be, for example, within the range of 5000 to 1000000, 10000 to 1000000, 10000 to 500000, or 90000 to 350000. The mass average molecular weight of the fluorine-containing polymer (A) is preferably within the range of 10000 to 750000, more preferably 40000 to 500000, and particularly preferably 70000 to 350000.

The lower limit of the mass average molecular weight of the fluorine-containing polymer (A) may be, for example, 5000 or more, preferably 10000 or more, more preferably 40000 or more, and particularly preferably 70000 or more. The upper limit of the mass average molecular weight of the fluorine-containing polymer (A) may be, for example, 1000000 or less, preferably 750000 or less, more preferably 500000 or less, and particularly preferably 350000 or less. The above upper and lower limits may be appropriately combined.

The mass average molecular weight of the fluorine-containing polymer (A) is a value determined by a gel permeation chromatography (GPC) method (in particular, the GPC method described later in the Examples).

### Polymerization Step

In the polymerization step, a fluorine-containing monomer (M) is polymerized in the presence of the fluorine-containing monomer (M), a polymerization initiator (B), and a solvent (D). The fluorine-containing polymer (A) is produced as a precipitate in the polymerization liquid.

### Starting Monomer

The starting monomer for use in the polymerization step contains at least a fluorine-containing monomer (M). The fluorine-containing monomer (M) is a monomer corresponding to the structural unit (A), which is the main component of the fluorine-containing polymer (A). As the fluorine-containing monomer (M), a single type may be used singly, or two or more types may be used in combination.

When the fluorine-containing polymer (A) to be produced comprises the fluoroolefin unit and/or other structural units in addition to the structural unit (A), the starting monomer may contain, in addition to the monomer (M), a fluoroolefin monomer corresponding to the fluoroolefin unit and/or monomers corresponding to the other structural units (which may be referred to as “other monomers” in the present specification).

Those skilled in the art would be able to understand that the polymerization of a specific monomer can produce a fluorine-containing polymer (A) comprising a structural unit corresponding to that monomer. Therefore, those skilled in the art would be able to select an appropriate monomer to produce a desired fluorine-containing polymer (A). Accordingly, those skilled in the art would be able to understand the monomer corresponding to the structural unit (A), the fluoroolefin monomer corresponding to the fluoroolefin unit, as well as the monomers corresponding to the other structural units.

For example, the monomer corresponding to the structural unit (A) is a fluorine-containing monomer (M) represented by formula (M):

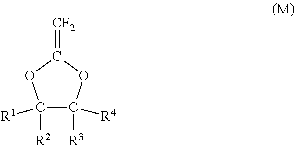

wherein R¹ to R⁴ each independently represent a fluorine atom, a C1-C5 perfluoroalkyl group, or a C1-C5 perfluoroalkoxy group.

For example, the monomer corresponding to the structural unit (A1) is a compound represented by formula (M1):

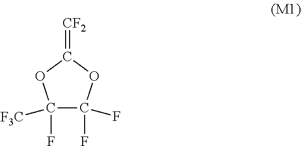

(which may be referred to as “the fluorine-containing monomer (M1)” in the present specification).

In the fluorine-containing monomer (M), there may be cases in which a compound (N) is mixed in. The amount of the compound (N) may be 0.0001 to 10 mass %, 0.0001 to 8 mass %, or 0.0001 to 6 mass %, based on the mass of the starting monomer. The compound (N) may be a single type or a combination of two or more types.

The compound (N) is a compound represented by formula (N):

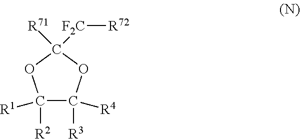

wherein R¹ to R⁴ each independently represent a fluorine atom, a C1-C5 perfluoroalkyl group, or a C1-C5 perfluoroalkoxy group, R⁷¹ and R⁷² each independently represent a hydrogen atom or a fluorine atom, and when one of R⁷¹ or R⁷² is a hydrogen atom, the other is a fluorine atom.

The details of R¹ to R⁴ are as defined above.

It is preferable that R⁷¹ is a hydrogen atom and R⁷² is a fluorine atom, or that R⁷¹ is a fluorine atom and R⁷² is a hydrogen atom.

A further preferred compound (N) is at least one compound selected from the group consisting of
a compound represented by formula (N1):

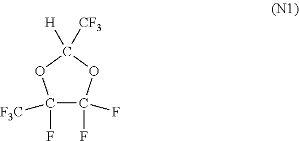 [SMILES: FC(F)(F)C1OC(F)(F)C(F)(C(F)(F)F)O1]

(which may be referred to as “the compound (N1)” in the present specification), and
a compound represented by formula (N2):

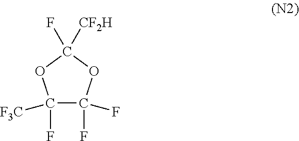 [SMILES: FC(F)C1(F)OC(F)(F)C(F)(C(F)(F)F)O1]

(which may be referred to as “the compound (N2) in the present specification).

The proportions (e.g., molar ratio) of the fluorine-containing monomer (M), fluoroolefin monomer, and other monomers in the starting monomer may be appropriately selected according to the proportions (e.g., molar ratio) of the structural units constituting the fluorine-containing polymer (A) to be produced. Accordingly, the details regarding the fluorine-containing monomer (M), fluoroolefin monomer, and other monomers can each be understood by those skilled in the art based on the descriptions above for the corresponding structural unit (A), fluoroolefin unit, and other structural unit.

The fluoroolefin monomer for use may be a monomer corresponding to the fluoroolefin unit described above. The fluoroolefin may be, for example, at least one selected from the group consisting of fluorine-containing perhaloolefin, vinylidene fluoride, trifluoroethylene, pentafluoropropylene, and 1,1,1,2-tetrafluoro-2-propylene. The fluoroolefin monomer may be preferably at least one selected from the group consisting of chlorotrifluoroethylene, tetrafluoroethylene, hexafluoropropylene, perfluoro(methyl vinyl ether), and perfluoro(propyl vinyl ether).

The fluorine-containing perhaloolefin may be at least one selected from the group consisting of chlorotrifluoroethylene, tetrafluoroethylene, hexafluoropropylene, perfluoro(methyl vinyl ether), perfluoro(ethyl vinyl ether), perfluoro(propyl vinyl ether), perfluoro(butyl vinyl ether), and perfluoro(2,2-dimethyl-1,3-dioxole).

### Polymerization Initiator (B)

The polymerization initiator (B) may be any polymerization initiator as long as the starting monomer can be polymerized. The polymerization initiator (B) may be selected from peroxides, azo compounds, and the like, and is preferably a peroxide. The polymerization initiators (B) may be used singly or in a combination of two or more.

The peroxide may be at least one compound selected from the group consisting of
a compound represented by formula (B1):

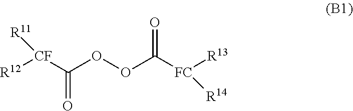

wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom (which may be referred to as “the compound (B1)” in the present specification),
a compound represented by formula (B2):

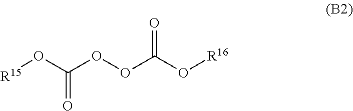

wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom (which may be referred to as “the compound (B2)” in the present specification), and
a compound represented by formula (B3):

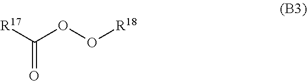

wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom (which may be referred to as “the compound (B3)” in the present specification) (which may be referred to as “the compound (B)” in the present specification).

In formula (B1), it is preferable that R¹¹ and R¹³ are identical and R¹² and R¹⁴ are identical, or that the ring structure formed by the linkage of R¹¹ and R¹² and the ring structure formed by the linkage of R¹³ and R¹⁴ are identical.

In formula (B1), R¹¹, R¹², R¹³, and R¹⁴ may each independently represent
a fluorine atom,
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5; or
R¹¹ and R¹² may be linked together to form a 4- to 8-membered perfluorocycloalkane ring, a perfluorobenzene ring, a perfluoronaphthalene ring, a perfluorooxolane ring, or a perfluorodioxolane ring, and
R¹³ and R¹⁴ may be linked together to form a 4- to 8-membered perfluorocycloalkane ring, a perfluorobenzene ring, a perfluoronaphthalene ring, a perfluorooxolane ring, or a perfluorodioxolane ring.

In formula (B1), R¹¹, R¹², R¹³, and R¹⁴ may each independently represent a fluorine atom,
—(CF₂)₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5; or
R¹¹ and R¹² may be linked together to form a 4- to 6-membered perfluorocycloalkane ring, a perfluorobenzene ring, or a perfluoronaphthalene ring, and
R¹³ and R¹⁴ may be linked together to form a 4- to 6-membered perfluorocycloalkane ring, a perfluorobenzene ring, or a perfluoronaphthalene ring.

In formula (B1), R¹¹, R¹², R¹³, and R¹⁴ may each independently represent
—(CF₂)₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5; or
R¹¹ and R¹² may be linked together to form a 4- to 6-membered perfluorocycloalkane ring, a perfluorobenzene ring, or a perfluoronaphthalene ring, and
R¹³ and R¹⁴ may be linked together to form a 4- to 6-membered perfluorocycloalkane ring, a perfluorobenzene ring, or a perfluoronaphthalene ring.

In formula (B1), R¹¹, R¹², R¹³, and R¹⁴ may each independently represent
a fluorine atom,
—(CF₂)ₙ₁CF₃ wherein n1 represents an integer of 0 to 2,
—(CF₂)ₙ₂CF₂H wherein n2 represents an integer of 0 to 2,
—O—(CF₂)ₙ₃CF₃ wherein n3 represents an integer of 0 to 2, or
—O—(CF₂)ₙ₄CF₂H wherein n4 represents an integer of 0 to 2; or
R¹¹ and R¹² may be linked together to form a 4- to 6-membered perfluorocycloalkane ring, a perfluorobenzene ring, or a perfluoronaphthalene ring, and
R¹³ and R¹⁴ may be linked together to form a 4- to 6-membered perfluorocycloalkane ring, a perfluorobenzene ring, or a perfluoronaphthalene ring.

In formula (B1), R¹¹, R¹², R¹³, and R¹⁴ may each independently represent:
—(CF₂)ₙ₁CF₃ wherein n1 represents an integer of 0 to 2,
—(CF₂)ₙ₂CF₂H wherein n2 represents an integer of 0 to 2,
—O—(CF₂)ₙ₃CF₃ wherein n3 represents an integer of 0 to 2, or
—O—(CF₂)ₙ₄CF₂H wherein n4 represents an integer of 0 to 2; or
R¹¹ and R¹² may be linked together to form a 4- to 6-membered perfluorocycloalkane ring, a perfluorobenzene ring, or a perfluoronaphthalene ring, and
R¹³ and R¹⁴ may be linked together to form a 4- to 6-membered perfluorocycloalkane ring, a perfluorobenzene ring, or a perfluoronaphthalene ring.

In formula (B1), R¹¹ and R¹³ may be fluorine atoms, and R¹² and R¹⁴ may each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5.

In formula (B2), it is preferable that R⁵ and R¹⁶ are identical.

In formula (B2), it is preferable that the alkyl group and the fluoroalkyl group do not have an etheric oxygen atom.

In formula (B2), the C4-C8 cyclic alkyl group may be cyclobutyl, cyclopentyl, cyclohexyl, cycloheptyl, or cyclooctyl.

In formula (B2), the C4-C8 cyclic fluoroalkyl group may be fluorocyclobutyl, fluorocyclopentyl, fluorocyclohexyl, fluorocycloheptyl, or fluorocyclooctyl.

In formula (B2), the C4-C8 cyclic fluoroalkyl group may be a C4-C6 cyclic fluoroalkyl group. The C4-C6 cyclic fluoroalkyl group may be fluorocyclobutyl, fluorocyclopentyl, or fluorocyclohexyl.

In formula (B2), the C4-C14 aryl group optionally having a fluorine atom may be phenyl optionally having a fluorine atom, naphthyl, anthryl, or phenanthryl.

In formula (B2), the C4-C14 aryl group optionally having a fluorine atom may be phenyl, naphthyl, anthryl, or phenanthryl.

In formula (B2), the C4-C14 aryl group optionally having a fluorine atom may be phenyl or naphthyl.

In formula (B2), R¹⁵ and R¹⁶ may each independently represent a C1-C7 linear or branched alkyl group, a C1-C7 linear or branched fluoroalkyl group, a C4-C8 cyclic alkyl group, a C4-C8 cyclic fluoroalkyl group, or a C4-C14 aryl group optionally having a fluorine atom. The alkyl group and the fluoroalkyl group each optionally have an etheric oxygen atom.

In formula (B2), R¹⁵ and R¹⁶ may each independently represent a C1-C7 linear or branched alkyl group, a C1-C7 linear or branched fluoroalkyl group, cyclobutyl, cyclopentyl, cyclohexyl, cycloheptyl, cyclooctyl, fluorocyclobutyl, fluorocyclopentyl, fluorocyclohexyl, fluorocycloheptyl, fluorocyclooctyl, phenyl optionally having a fluorine atom, naphthyl optionally having a fluorine atom, anthryl optionally having a fluorine atom, or phenanthryl optionally having a fluorine atom.

In formula (B2), R¹⁵ and R¹⁶ may each independently represent a C1-C7 linear or branched alkyl group, a C1-C7 linear or branched fluoroalkyl group, cyclobutyl, cyclopentyl, cyclohexyl, fluorocyclobutyl, fluorocyclopentyl, fluorocyclohexyl, phenyl, naphthyl, phenyl having a fluorine atom, or naphthyl having a fluorine atom.

In formula (B2), R¹⁵ and R¹⁶ may each independently represent a C1-C7 linear or branched alkyl group, or a C1-C7 linear or branched fluoroalkyl group.

In formula (B3), it is preferable that R¹⁷ and R¹⁸ are not identical.

In formula (B3), when R¹⁷ is an alkyl group or a fluoroalkyl group, it is preferable that an etheric oxygen atom is not present.

In formula (B3), the C4-C8 cyclic alkyl group may be cyclobutyl, cyclopentyl, cyclohexyl, cycloheptyl, or cyclooctyl.

In formula (B3), the C4-C8 cyclic fluoroalkyl group may be fluorocyclobutyl, fluorocyclopentyl, fluorocyclohexyl, fluorocycloheptyl, or fluorocyclooctyl.

In formula (B3), the C4-C8 cyclic fluoroalkyl group may be a C4-C6 cyclic fluoroalkyl group. The C4-C6 cyclic fluoroalkyl group may be fluorocyclobutyl, fluorocyclopentyl, or fluorocyclohexyl.

In formula (B3), the C4-C14 aryl group optionally having a fluorine atom may be phenyl optionally having a fluorine atom, naphthyl, anthryl, or phenanthryl.

In formula (B3), the C4-C14 aryl group optionally having a fluorine atom may be phenyl, naphthyl, anthryl, or phenanthryl.

In formula (B3), the C4-C14 aryl group optionally having a fluorine atom may be phenyl optionally having a fluorine atom or naphthyl.

In formula (B3), the C4-C14 aryl group optionally having a fluorine atom may be phenyl or naphthyl.

In formula (B3), R¹⁷ and R¹⁸ may each independently represent a C1-C9 linear or branched alkyl group, a C1-C9 linear or branched fluoroalkyl group, a C4-C8 cyclic alkyl group, a C4-C8 cyclic fluoroalkyl group, or a C4-C14 aryl group optionally having a fluorine atom. The alkyl group and the fluoroalkyl group each optionally have an etheric oxygen atom.

In formula (B3), R¹⁷ and R¹⁸ may each independently represent a C1-C9 linear or branched alkyl group, a C1-C9 linear or branched fluoroalkyl group, cyclobutyl, cyclopentyl, cyclohexyl, cycloheptyl, cyclooctyl, fluorocyclobutyl, fluorocyclopentyl, fluorocyclohexyl, fluorocycloheptyl, fluorocyclooctyl, phenyl optionally having a fluorine atom, naphthyl optionally having a fluorine atom, anthryl optionally having a fluorine atom, or phenanthryl optionally having a fluorine atom. The alkyl group and the fluoroalkyl group each optionally have an etheric oxygen atom.

In formula (B3), R¹⁷ and R¹⁸ may each independently represent a C1-C9 linear or branched alkyl group, a C1-C9 linear or branched fluoroalkyl group, cyclobutyl, cyclopentyl, cyclohexyl, fluorocyclobutyl, fluorocyclopentyl, fluorocyclohexyl, phenyl, naphthyl, phenyl having a fluorine atom, or naphthyl having a fluorine atom. The alkyl group and the fluoroalkyl group each optionally have an etheric oxygen atom.

In formula (B3), R¹⁷ may be a C1-C6 linear alkyl group, a C3-C9 branched alkyl group, a C1-C6 linear fluoroalkyl group, a C3-C6 branched fluoroalkyl group, or phenyl.

In formula (B3), R¹⁷ may be methyl, tert-butyl, tert-nonyl, trifluoromethyl, perfluoroisopentyl, or phenyl.

In formula (B3), R¹⁸ may be a C1-C6 linear alkyl group, a C3-C6 branched alkyl group, a C1-C6 linear fluoroalkyl group, or a C3-C6 branched fluoroalkyl group. The alkyl group and the fluoroalkyl group each optionally have an etheric oxygen atom.

In formula (B3), R¹⁸ may be tert-butyl, tert-hexyl, or trifluoromethyl.

In formula (B3), R¹⁷ may be methyl, tert-butyl, tert-nonyl, trifluoromethyl, perfluoroisopentyl, or phenyl, and R¹⁸ may be tert-butyl, tert-hexyl, or trifluoromethyl.

Examples of the compound (B1) include compounds represented by the structural formulas shown below. In the following structural formulas, m1 represents an integer of 0 to 6, m2 represents an integer of 0 to 6, m3 represents an integer of 0 to 2, and m4 represents an integer of 0 to 2, and m3 and m4 may be the same or different.

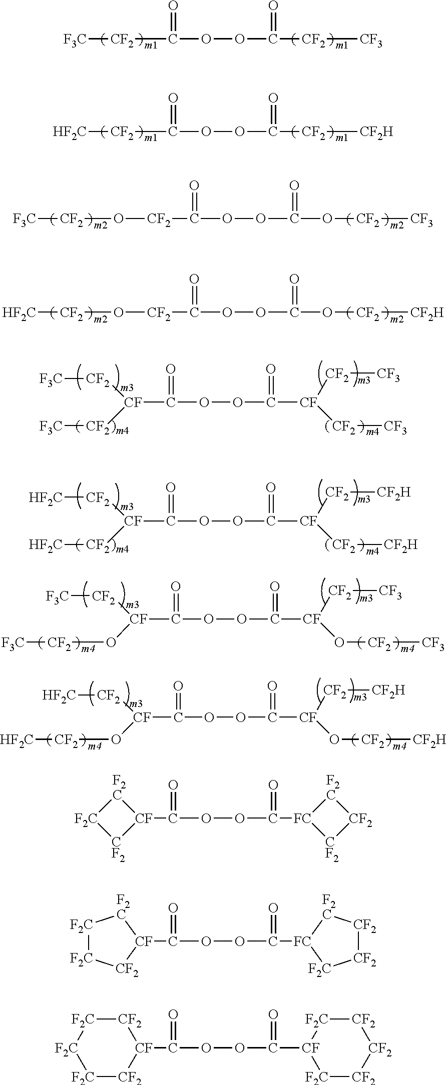

Preferred examples of the compound (B1) include bis(2,2,3,3,4,4,5,5,6,6,7,7-dodecafluoro-1-oxoheptyl) peroxide (DHP) and bis(cyclohexylcarbonyl) peroxide.

Examples of the compound (B2) include di-sec-butyl peroxydicarbonate, di-n-propyl peroxydicarbonate (NPP), diisopropyl peroxydicarbonate, di-2-ethylhexyl peroxydicarbonate, ditrifluoromethyl peroxydicarbonate, dipentafluoroethyl peroxydicarbonate, diheptafluoro-n-propyl peroxydicarbonate, and diheptafluoroisopropyl peroxydicarbonate.

Preferred examples of the compound (B2) include diisopropyl peroxydicarbonate, di-n-propyl peroxydicarbonate (NPP), and ditrifluoromethyl peroxydicarbonate.

Examples of the compound (B3) include tert-butyl peroxyacetate (TBPOA), tert-butyl peroxypivalate, tert-hexyl peroxypivalate, tert-butyl peroxyneodecanoate, tert-butyl peroxybenzoate, trifluoromethyl-2,2,2-trifluoromethylethane peroxoate (CF₃COOOCF₃), and heptafluoroisopropyl-2,2,2-trifluoromethylethane peroxoate ((CF₃)₂CFCOOOCF₃).

Preferred examples of the compound (B3) include tert-butyl peroxyacetate (TBPOA), tert-butyl peroxybenzoate, and trifluoromethyl-2,2,2-trifluoromethylethane peroxoate (CF₃COOOCF₃).

### Decomposed Product (C) of Polymerization Initiator (B)

A decomposed product (C) of the polymerization initiator (B) is produced by the decomposition of the polymerization initiator (B) during the polymerization step. After the polymerization reaction, the decomposed product (C) is contained in both the polymerization reaction liquid and precipitate. The decomposed product (C) present in the polymerization reaction liquid is separated from the precipitate during the filtration step. The decomposed product (C) may be used singly or in a combination of two or more.

The decomposed product (C) may be at least one compound selected from the group consisting of a compound represented by formula (C1):

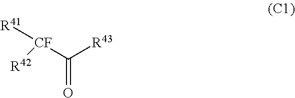

wherein R⁴¹ and R⁴² each independently represent a fluorine atom, a C1-C20 alkyl group, or a C1-C20 fluoroalkyl group, the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group (which may be referred to as “the compound (C1)” in the present specification); a compound represented by formula (C2)

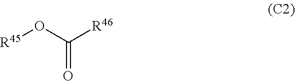

wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group (which may be referred to as “the compound (C2)” in the present specification); and a compound represented by formula (C3):

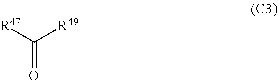

wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group (which may be referred to as “the compound (C3)” in the present specification) (which may be referred to as “the compound (C) in the present specification).

In formula (C1), R⁴¹ and R⁴² may each independently represent
a fluorine atom,
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, or
R⁴¹ and R⁴² may be linked together to form a 4- to 8-membered perfluorocycloalkane ring, a perfluorobenzene ring, a perfluoronaphthalene ring, a perfluorooxolane ring, or a perfluorodioxolane ring, and
R⁴³ may be a fluorine atom or a hydroxy group.

In formula (C1), R⁴¹ and R⁴² may each independently represent
a fluorine atom,
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, or
R⁴¹ and R⁴² may be linked together to form a 4- to 6-membered perfluorocycloalkane ring, a perfluorobenzene ring, or a perfluoronaphthalene ring, and
R⁴³ may be a fluorine atom or a hydroxy group.

In the formula (C1), R⁴¹ and R⁴² may each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, or
R⁴¹ and R⁴² may be linked together to form a 4- to 6-membered perfluorocycloalkane ring, a perfluorobenzene ring, or a perfluoronaphthalene ring, and
R⁴³ may be a fluorine atom or a hydroxy group.

In formula (C1), R⁴¹ and R⁴² may each independently represent
a fluorine atom,
—(CF₂)ₙ₁CF₃ wherein n1 represents an integer of 0 to 2,
—(CF₂)ₙ₂CF₂H wherein n2 represents an integer of 0 to 2,
—O—(CF₂)ₙ₃CF₃ wherein n3 represents an integer of 0 to 2, or
—O—(CF₂)ₙ₄CF₂H wherein n4 represents an integer of 0 to 2, or
R⁴¹ and R⁴² may be linked together to form a 4- to 6-membered perfluorocycloalkane ring, a perfluorobenzene ring, or a perfluoronaphthalene ring, and
R⁴³ may be a fluorine atom or a hydroxy group.

In formula (C1), R⁴¹ and R⁴² may each independently represent
—(CF₂)ₙ₁CF₃ wherein n1 represents an integer of 0 to 2,
—(CF₂)ₙ₂CF₂H wherein n2 represents an integer of 0 to 2,
—O—(CF₂)ₙ₃CF₃ wherein n3 represents an integer of 0 to 2, or
—O—(CF₂)ₙ₄CF₂H wherein n4 represents an integer of 0 to 2, or
R⁴¹ and R⁴² may be linked together to form a 4- to 6-membered perfluorocycloalkane ring, a perfluorobenzene ring, or a perfluoronaphthalene ring, and,
R⁴³ may be a fluorine atom or a hydroxy group.

In formula (C1), R⁴¹ may be a fluorine atom, R⁴² may be
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ may be a fluorine atom or a hydroxy group.

In formula (C2), it is preferable that the alkyl group and the fluoroalkyl group do not have an etheric oxygen atom.

In formula (C2), the C4-C8 cyclic alkyl group may be cyclobutyl, cyclopentyl, cyclohexyl, cycloheptyl, or cyclooctyl.

In formula (C2), the C4-C8 cyclic fluoroalkyl group may be fluorocyclobutyl, fluorocyclopentyl, fluorocyclohexyl, fluorocycloheptyl, or fluorocyclooctyl.

In formula (C2), the C4-C8 cyclic fluoroalkyl group may be a C4-C6 cyclic fluoroalkyl group. The C4-C6 cyclic fluoroalkyl group may be fluorocyclobutyl, fluorocyclopentyl, or fluorocyclohexyl.

In formula (C2), the C4-C14 aryl group optionally having a fluorine atom may be phenyl optionally having a fluorine atom, naphthyl, anthryl, or phenanthryl.

In formula (C2), the C4-C14 aryl group optionally having a fluorine atom may be phenyl, naphthyl, anthryl, or phenanthryl.

In formula (C2), the C4-C14 aryl group optionally having a fluorine atom may be phenyl or naphthyl.

In formula (C2), R⁴⁵ may each be a C1-C7 linear or branched alkyl group, a C1-C7 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁶ may be a fluorine atom or a hydroxy group.

In formula (C2), R⁴⁵ may be a C1-C7 linear or branched alkyl group, a C1-C7 linear or branched fluoroalkyl group, cyclobutyl, cyclopentyl, cyclohexyl, cycloheptyl, cyclooctyl, fluorocyclobutyl, fluorocyclopentyl, fluorocyclohexyl, fluorocycloheptyl, fluorocyclooctyl, phenyl optionally having a fluorine atom, naphthyl optionally having a fluorine atom, anthryl optionally having a fluorine atom, or phenanthryl optionally having a fluorine atom, and R⁴⁶ may be a fluorine atom or a hydroxy group.

In formula (C2), R⁴⁵ may be a C1-C7 linear or branched alkyl group, a C1-C7 linear or branched fluoroalkyl group, cyclobutyl, cyclopentyl, cyclohexyl, fluorocyclobutyl, fluorocyclopentyl, fluorocyclohexyl, phenyl, naphthyl, phenyl having a fluorine atom, or naphthyl having a fluorine atom, and R⁴⁶ may be a fluorine atom or a hydroxy group.

In formula (C2), R⁴⁵ may be a C1-C7 linear or branched alkyl group, or a C1-C7 linear or branched fluoroalkyl group, and R⁴⁶ may be a fluorine atom or a hydroxy group.

In formula (C3), when R⁴⁷ is an alkyl group or a fluoroalkyl group, it is preferable that an etheric oxygen atom is not present.

In formula (C3), the C4-C8 cyclic alkyl group may be cyclobutyl, cyclopentyl, cyclohexyl, cycloheptyl, or cyclooctyl.

In formula (C3), the C4-C8 cyclic fluoroalkyl group may be fluorocyclobutyl, fluorocyclopentyl, fluorocyclohexyl, fluorocycloheptyl, or fluorocyclooctyl.

In formula (C3), the C4-C8 cyclic fluoroalkyl group may be a C4-C6 cyclic fluoroalkyl group. The C4-C6 cyclic fluoroalkyl group may be fluorocyclobutyl, fluorocyclopentyl, or fluorocyclohexyl.

In formula (C3), the C4-C14 aryl group optionally having a fluorine atom may be phenyl optionally having a fluorine atom, naphthyl, anthryl, or phenanthryl.

In formula (C3), the C4-C14 aryl group optionally having a fluorine atom may be phenyl, naphthyl, anthryl, or phenanthryl.

In formula (C3), the C4-C14 aryl group optionally having a fluorine atom may be phenyl or naphthyl.

In formula (C3), the C4-C14 aryl group optionally having a fluorine atom may be phenyl or naphthyl.

In formula (C3), R⁴⁷ may be a C1-C9 linear or branched alkyl group, a C1-C9 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ may be a fluorine atom or a hydroxy group.

In formula (C3), R⁴⁷ may be a C1-C9 linear or branched alkyl group, a C1-C9 linear or branched fluoroalkyl group, cyclobutyl, cyclopentyl, cyclohexyl, cycloheptyl, cyclooctyl, fluorocyclobutyl, fluorocyclopentyl, fluorocyclohexyl, fluorocycloheptyl, fluorocyclooctyl, phenyl optionally having a fluorine atom, naphthyl optionally having a fluorine atom, anthryl optionally having a fluorine atom, or phenanthryl optionally having a fluorine atom, the alkyl group and the fluoroalkyl group each optionally have an etheric oxygen atom, and R⁴⁹ may be a fluorine atom or a hydroxy group.

In formula (C3), R⁴⁷ may be a C1-C9 linear or branched alkyl group, a C1-C9 linear or branched fluoroalkyl group, cyclobutyl, cyclopentyl, cyclohexyl, fluorocyclobutyl, fluorocyclopentyl, fluorocyclohexyl, phenyl, naphthyl, phenyl having a fluorine atom, or naphthyl having a fluorine atom, the alkyl group and the fluoroalkyl group each optionally have an etheric oxygen atom, and R⁴⁹ may be a fluorine atom or a hydroxy group.

In formula (C3), R⁴⁷ may be a C1-C6 linear alkyl group, a C3-C9 branched alkyl group, a C1-C6 linear fluoroalkyl group, a C3-C6 branched fluoroalkyl group, or phenyl, and R⁴⁹ may be a fluorine atom or a hydroxy group.

In formula (C3), R⁴⁷ may be methyl, tert-butyl, tert-nonyl, trifluoromethyl, perfluoroisopentyl, or phenyl, and R⁴⁹ may be a fluorine atom or a hydroxy group.

In formula (C3), R⁴⁹ may be a C1-C6 linear alkyl group, a C3-C6 branched alkyl group, a C1-C6 linear fluoroalkyl group, or a C3-C6 branched fluoroalkyl group, the alkyl group and the fluoroalkyl group each optionally have an etheric oxygen atom, and R⁴⁹ may be a fluorine atom or a hydroxy group.

In formula (C3), R⁴⁹ may be tert-butyl, tert-hexyl, or trifluoromethyl.

In formula (C3), R⁴⁷ may be methyl, tert-butyl, tert-nonyl, trifluoromethyl, perfluoroisopentyl, or phenyl, and R⁴⁹ may be a fluorine atom or a hydroxy group.

Examples of the compound (C1) include compounds represented by the structural formulas shown below. In the following structural formulas, m1 represents an integer of 0 to 6, m2 represents an integer of 0 to 6, m3 represents an integer of 0 to 2, m4 represents an integer of 0 to 2, m3 and m4 may be identical or different, and R⁴³ represents a fluorine atom or a hydroxy group.

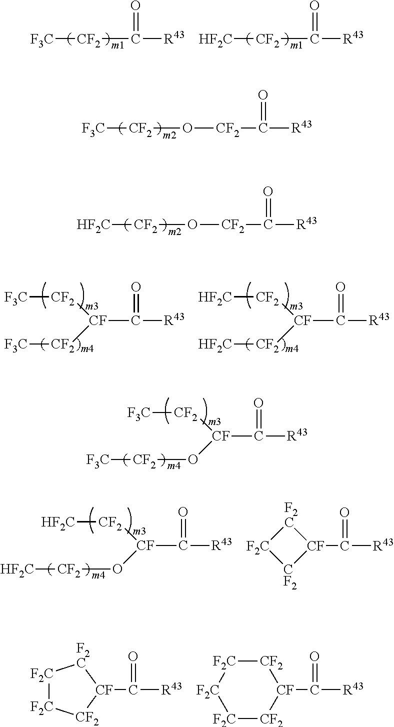

Preferred examples of the compound (C1) include 2,2,3,3,4,4,5,5,6,6,7,7-dodecafluoro-1-oxoheptylcarboxylic acid, 2,2,3,3,4,4,5,5,6,6,7,7-dodecafluoro-1-oxoheptylcarbonyl fluoride, cyclohexylcarboxylic acid, and cyclohexylcarbonyl fluoride.

Examples of the compound (C2) include n-propyloxycarboxylic acid, n-propyloxycarbonyl fluoride, sec-butyloxycarboxylic acid, sec-butyloxycarbonyl fluoride, isopropyloxycarboxylic acid, isopropyloxycarbonyl fluoride, 2-ethylhexyloxycarboxylic acid, 2-ethylhexyloxycarbonyl fluoride, trifluoromethyloxycarboxylic acid, trifluoromethyloxycarbonyl fluoride, pentafluoroethyloxycarboxylic acid, pentafluoroethyloxycarbonyl fluoride, heptafluoro-n-propyloxycarboxylic acid, heptafluoro-n-propyloxycarbonyl fluoride, heptafluoroisopropyloxycarboxylic acid, and heptafluoroisopropyloxycarbonyl fluoride.

Preferred examples of the compound (C2) include n-propyloxycarboxylic acid, n-propyloxycarbonyl fluoride, isopropyloxycarboxylic acid, isopropyloxycarbonyl fluoride, trifluoromethyloxycarboxylic acid, and trifluoromethyloxycarbonyl fluoride.

Examples of the compound (C3) include acetic acid, methyl carbonyl fluoride, tert-butylcarboxylic acid, tert-butylcarbonyl fluoride, neononylcarboxylic acid, neononylcarbonyl fluoride, benzoic acid, phenylcarbonyl fluoride, heptafluoroisopentylcarboxylic acid, heptafluoroisopentylcarbonyl fluoride, trifluorocarboxylic acid, and trifluorocarbonyl fluoride.

Preferred examples of the compound (C3) include acetic acid, methylcarbonyl fluoride, benzoic acid, phenylcarbonyl fluoride, trifluorocarboxylic acid, and trifluorocarbonyl fluoride.

### Solvent (D)

The fluorine-containing monomer (M) is polymerized in the solvent (D). The solvent may be any solvent that can dissolve the starting monomer and precipitate the fluorine-containing polymer (A). Therefore, the solvent (D) may be any known solvent for use in the production of the fluorine-containing polymer (A) by precipitation polymerization. The solvents (D) may be used singly or in a combination of two or more.

The solvent (D) may be an aprotic solvent. Examples of aprotic solvents include nonperfluoro solvents.

The “nonperfluoro solvent” refers to an aprotic solvent that contains at least fluorine, carbon, and hydrogen atoms. Examples of nonperfluoro solvents include hydrofluorocarbons, hydrofluoroethers, fluorine-containing alcohols, and olefin compounds containing at least one chlorine atom. The nonperfluoro solvents may be used singly or in a combination of two or more.

The hydrofluorocarbon is, for example, a C3-C8 hydrofluorocarbon. Examples of hydrofluorocarbons include CF₃CH₂CF₂H, CF₃CH₂CF₂CH₃, CF₃CHFCHFC₂F₅, 1,1,2,2,3,3,4-heptafluorocyclopentane, CF₃CF₂CF₂CF₂CH₂CH₃, CF₃CF₂CF₂CF₂CF₂CHF₂, and CF₃CF₂CF₂CF₂CF₂CF₂CH₂CH₃.

Preferred examples of hydrofluorocarbons include 1,1,2,2,3,3,4-heptafluorocyclopentane.

The hydrofluoroether is, for example, a fluorine-containing ether.

The hydrofluoroether preferably has a global warming potential (GWP) of 600 or less, more preferably 400 or less, and particularly preferably 300 or less. The lower limit of the global warming potential (GWP) of the hydrofluoroether may be 1 or more, or 5 or more.

Examples of hydrofluoroethers include CF₃CF₂CF₂CF₂OCH₃, CF₃CF₂CF(CF₃) OCH₃, CF₃CF(CF₃) CF₂OCH₃, CF₃CF₂CF₂CF₂OC₂H₅, CF₃CH₂OCF₂CHF₂, (CF₃)₂CHOCH₃, (CF₃)₂CFOCH₃, CHF₂CF₂OCH₂CF₃, CHF₂CF₂CH₂OCF₂CHF₂, CF₃CHFCF₂OCH₃, CF₃CHFCF₂OCF₃, trifluoromethyl 1,2,2,2-tetrafluoroethyl ether (HFE-227me), difluoromethyl 1,1,2,2,2-pentafluoroethyl ether (HFE-227mc), trifluoromethyl 1,1,2,2-tetrafluoroethyl ether (HFE-227pc), difluoromethyl 2,2,2-trifluoroethyl ether (HFE-245mf), 2,2-difluoroethyltrifluoromethyl ether (HFE-245pf), 1,1,2,3,3-hexafluoropropyl methyl ether (CF₃CHFCF₂OCH₃), 1,1,1,2,2-tetrafluoroethyl 2,2,2-trifluoroethyl ether (CHF₂CF₂OCH₂CF₃), and 1,1,1,3,3,3-hexafluoro-2-methoxypropane ((CF₃)₂CHOCH₃).

Preferred examples of hydrofluoroethers include CF₃CH₂OCF₂CHF₂, 1,1,2,3,3-hexafluoropropyl methyl ether (CF₃CHFCF₂OCH₃; HFE-356mec), 1,1,1,2,2-tetrafluoroethyl 2,2,2-trifluoroethyl ether (CHF₂CF₂OCH₂CF₃), and 1,1,1,3,3,3-hexafluoro-2-methoxypropane ((CF₃)₂CHOCH₃).

The “fluorine-containing alcohol” refers to a compound in which at least one hydrogen atom in the alcohol molecule is replaced by a fluorine atom.

Examples of fluorine-containing alcohols include CF₃CH₂OH, CF₃CF₂CH₂OH, CF₃ (CF₂)₂CH₂OH, CF₃ (CF₂)₃(CH₂)₂OH, CF₃ (CF₂)₃(CH₂)₃OH, CF₃ (CF₂)₅(CH₂)₂OH, CF₃ (CF₂)₅(CH₂)₃OH, CF₂HCF₂CH₂OH, CF₂H(CF₂)₃CH₂OH, CF₂H(CF₂)₅CH₂OH, (CF₃)₂CHOH, CF₃CHFCF₂CH₂OH, HOCH₂ (CF₂)₄CH₂OH, and HOCH₂ (CF₂)₆CH₂OH.

Preferred examples of fluorine-containing alcohols include CF₃CF₂CH₂OH, CF₂HCF₂CH₂OH, CF₂H(CF₂)₃CH₂OH, and (CF₃)₂CHOH.

The olefin compound containing at least one chlorine atom is a C2-C4 (preferably C2-C3) olefin compound containing at least one chlorine atom in its structure. The olefin compound containing at least one chlorine atom is a compound in which at least one of the hydrogen atoms bonded to the carbon atoms in a C2-C4 hydrocarbon having one or two (preferably one) double bonds is replaced by chlorine.

The number of chlorine atoms is one to the maximum substitutable number. The number of chlorine atoms may be, for example, one, two, three, four, or five.

The olefin compound containing at least one chlorine atom may contain at least one (e.g., one, two, three, four, or five) fluorine atom.

Examples of olefin compounds containing at least one chlorine atom include CH₂═CHCl, CHCl═CHCl, CCl₂═CHCl, CCl₂=CCl₂, CF₃CH═CHCl, CHF₂CF═CHCl, CFH₂CF═CHCl, CF₃CCl=CFCl, CF₂HCl=CFCl, and CFH₂Cl=CFCl.

Preferred examples of olefin compounds containing at least one chlorine atom include CHCl═CHCl, CHF₂CF═CHCl, CF₃CH═CHCl, and CF₃CCl=CFCl.

The solvent (D) is preferably a hydrofluoroether or a fluorine-containing alcohol because they have less environmental impact during use and can be easily distilled off from the polymer.

The solvent (D) may have a global warming potential (GWP) of, for example, 600 or less, or 400 or less. The solvent (D) preferably has a global warming potential (GWP) of 375 or less, more preferably 350 or less, and particularly preferably 0. The lower limit of the global warming potential (GWP) of the solvent (D) may be 1 or more, or 5 or more.

The amount of the fluorine-containing monomer (M) used in the polymerization step can be appropriately determined according to, for example, the proportion of the structural unit (A) in the desired fluorine-containing polymer (A). For example, the amount of the fluorine-containing monomer (M) is 50 mol % or more, preferably 80 mol % or more, more preferably 90 mol % or more, and particularly preferably 100 mol %, based on the total number of moles of the starting monomer.

In addition to the fluorine-containing monomer (M), when the fluoroolefin monomer and/or other monomers are used, the amounts of these monomers may be appropriately determined according to, for example, the proportions of the fluoroolefin unit and other structural units in the desired fluorine-containing polymer (A). For example, the amount of the fluoroolefin monomer may be 50 mol % or less, preferably 20 mol % or less, more preferably 10 mol % or less, and particularly preferably 0 mol %, based on the total number of moles of the starting monomers. The amount of the other monomers may be 0 to 20 mol %, 0 to 10 mol %, or the like, preferably 20 mol % or less, and more preferably 10 mol % or less, based on the total number of moles of the starting monomers.

The amount of the polymerization initiator (B) for use in the polymerization step may be, for example, within the range of 0.0001 g to 0.05 g, preferably 0.0001 g to 0.01 g, and more preferably 0.0005 g to 0.008 g, per 1 g of all of the starting monomers.

The amount of the solvent (D) for use in the polymerization step may be, for example, within the range of 20 mass % to 300 mass %, and preferably 35 mass % to 250 mass %, and more preferably 50 mass % to 300 mass %, based on the total amount of the starting monomers taken as 100 mass %.

The temperature of the polymerization reaction may be, for example, within the range of −10° C. to 160° C., preferably 0° C. to 160° C., and more preferably 0° C. to 100° C.

The polymerization reaction may be performed at a temperature that is not greater than 20° C. higher than the boiling point of the fluorine-containing monomer (M) or the boiling point of the solvent (D), whichever is lower, and that is not greater than 20° C. higher than the 10-hour half-life temperature of the polymerization initiator. In this case, the lower limit of the temperature may be, for example, −10° C., and preferably 0° C.

The reaction time of the polymerization reaction is not particularly limited as long as the fluorine-containing polymer (A) is produced. The reaction time may be preferably within the range of 0.5 hours to 72 hours, more preferably 1 hour to 48 hours, and even more preferably 3 hours to 30 hours.

The polymerization reaction can be performed in the presence or absence of an inert gas (e.g., nitrogen gas), and preferably in the presence of an inert gas.

The polymerization reaction can be performed under reduced pressure, atmospheric pressure, or increased pressure.

In the polymerization step, the order of the addition and mixing of the starting monomer, polymerization initiator (B), and solvent (D) is not particularly limited. For example, the step may be performed by adding the starting monomer to the solvent (D) containing the polymerization initiator (A) or by adding the polymerization initiator to the solvent (D) containing the starting monomer.

The fluorine-containing polymer (A) produced in the polymerization reaction can be isolated or purified, if desired, by a conventional method, such as extraction, dissolution, concentration, filtration, precipitation, dehydration, adsorption, or chromatography, or a combination of these methods. Preferred is filtration, which is simple and easy.

### Filtration Step

The production method of the present disclosure may comprise a filtration step. In the filtration step, the precipitate and a polymerization liquid produced in the polymerization step can be filtered to remove the polymerization liquid to thereby obtain a fluorine-containing polymer (A). The filtered solid is a fluorine-containing polymer (A) with a very small amount of contamination of the polymerization initiator (B) and/or its decomposed product (C). To further reduce the amount of contamination, the filtered solid may be washed with a solvent (e.g., polymerization solvent). Thus, in the production method of the present disclosure, a fluorine-containing polymer (A) with a very small amount of contamination of the polymerization initiator (B) and/or its decomposed product (C) can be obtained by performing only a simple filtration step, or a washing step of washing the solid obtained by filtration with a solvent (e.g., polymerization solvent), after the polymerization step.

Usable filter materials are resin products, such as polyethylene, polypropylene, polyester, nylon, and polytetrafluoroethylene; cotton fiber products, such as cellulose; and metal products, such as SUS. The pore size of the filter material may be 1 μm, 10 μm, 100 μm, 300 μm, 500 μm, or 1000 μm. The pore size may be a numerical range obtained by appropriately combining two of these numerical values. The pore size may be, for example, 1 to 1000 μm, 1 to 500 μm, 1 to 300 μm, 1 to 100 μm, 10 to 1000 μm, 100 to 1000 μm, or 300 to 1000 μm. The filtration step can be performed under reduced pressure, atmospheric pressure, or increased pressure.

### Hansen Solubility Parameters

The Hildebrand solubility parameter (δ) is a physical property value defined as the square root of the cohesive energy density, is one of the indices that indicate the solubility behavior of a substance, and is abbreviated as “SP.” Parameters obtained by dividing SP into three components, i.e., dispersion parameter (δ_{D}), polarity parameter (δ_{P}), and hydrogen bonding parameter (δ_{H}), and taking into account the polarity of the substance are Hansen solubility parameters, which are abbreviated as “HSPs.” The relationship between them is expressed as “δ²=δ_{D}²+δ_{P}²+δ_{H}².”

In the production method of the present disclosure, the Hansen solubility parameters (which may be referred to as “the solubility parameters” or “HSPs” in the present specification) of the fluorine-containing polymer (A), polymerization initiator (B), decomposed product (C), and solvent (D) may have a specific relationship. This can reduce the amount of the polymerization initiator (B) and/or its decomposed product (C) mixed into the fluorine-containing polymer (A) produced by polymerization.

The HSPs of the fluorine-containing polymer (A), polymerization initiator (B), decomposed product (C), and solvent (D) may be either values experimentally determined by dissolution testing (including literature values and database values) or values estimated from the chemical structures of the substances using HSP determination software (e.g., HSPiP).

### Solubility Index R

The solubility index R is calculated according to the following equation and corresponds to the distance between the HSPs of element 1 and the HSPs of element 2. If this distance is close, that is, if R is small, element 1 and element 2 are considered to be in a soluble relationship.

$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$

wherein δD₁, δP₁, and δH₁ respectively represent a dispersion parameter, a polarity parameter, and a hydrogen bonding parameter in the Hansen solubility parameters of element 1, and δD₂, δP₂, and δH₂ respectively represent a dispersion parameter, a polarity parameter, and a hydrogen bonding parameter in the Hansen solubility parameters of element 2.

The value (R_{A}) of the solubility index R, calculated based on the HSPs of the solvent (D) and the HSPs of the fluorine-containing polymer (A), may be 4 or more.

When the mass percentage of fluorine atoms in the polymerization initiator (B) is less than 50 mass %, the value (R_{B}) of the solubility index R, calculated based on the HSPs of the solvent (D) and the HSPs of the polymerization initiator (B), may be 9 or less, and preferably 8 or less.

When the mass percentage of fluorine atoms in the polymerization initiator (B) is 50 mass % or more, the value (R_{B}) of the solubility index R, calculated based on the HSPs of the solvent (D) and the HSPs of the polymerization initiator (B), may be 6 or less, and preferably 5 or less.

When the mass percentage of fluorine atoms in the decomposed product (C) is less than 50 mass %, the value (R_{C}) of the solubility index R, calculated based on the HSPs of the solvent (D) and the HSPs of the decomposed product (C), may be 9 or less, and preferably 8 or less.

When the mass percentage of fluorine atoms in the decomposed product (C) is 50 mass % or more, the value (R_{C}) of the solubility index R, calculated based on the HSPs of the solvent (D) and the HSPs of the decomposed product (C), may be 6 or less, and preferably 5 or less.

The mass percentage (mass %) of fluorine atoms in a substance is the percentage of the mass of fluorine atoms (atomic weight: 19.00) in the molecular weight of the substance. For example, the mass percentage of fluorine atoms in tetrafluoroethylene (molecular weight: 100.02; CF₂=CF₂) is 75.98%

$$( 75 \text{.98} = ( ( 19 \text{.00} \times 4 ) / 10 \; 0.02 ) \times 100 ) .$$

The production method of the present disclosure is suitable as the method for producing compositions (1) and (2) described below.

### Composition (1)

An embodiment of the present disclosure is a composition comprising a fluorine-containing polymer (A) and at least one compound (BC) selected from the group consisting of a polymerization initiator (B) and a decomposed product (C) of the polymerization initiator (B) (which may be referred to as “the composition (1)” in the present specification). Since the composition (1) has a low content of the compound (BC), the composition (1) is useful as a source of the fluorine-containing polymer (A) with high ultraviolet light permeability (e.g., a source of the fluorine-containing polymer (A) used in products through which ultraviolet light passes (preferably lenses for ultraviolet-light-emitting elements)).

The fluorine-containing polymer (A) is preferably produced by the method for producing a fluorine-containing polymer (A) of the present disclosure because the content of a polymerization initiator and/or its decomposed product becomes low. The fluorine-containing polymer (A) may also be produced by a known production method and purified so that the content of a polymerization initiator and/or its decomposed product becomes low. As such a known production method, for example, it can be synthesized by polymerizing monomers corresponding to the structural units of the fluorine-containing polymer (A). The polymerization method may be, for example, radical polymerization, bulk polymerization, precipitation polymerization, solution polymerization, suspension polymerization, or emulsion polymerization. A preferred known production method is precipitation polymerization, in terms of the high permeability of the fluorine-containing polymer (A).

The content of the fluorine-containing polymer (A) in the composition (1) can be 95 mass % or more based on the mass of the composition (1). The content can be preferably 97 to 100 mass %, more preferably 98 to 100 mass %, and particularly preferably 99 to 100 mass %.

The content of the compound (BC) in the composition (1) can be 0.1 to 100 mass ppm, preferably 0.1 to 90 mass ppm, more preferably 0.1 to 80 mass ppm, and particularly preferably 0.1 to 70 mass ppm, based on the mass of the composition (1).

The composition (1) may contain other components in addition to the fluorine-containing polymer (A) and the compound (BC), but preferably does not contain other components. Examples of other components include metals such as Fe and Ni, the residual solvent, and the like. The content of other components in the composition (1) may be 0 to 0.1 mass % or 0 to 0.01 mass %.

The composition (1) can be produced by the production method of the present disclosure. For example, the solid obtained in the filtration step or a washed product obtained by washing the solid with a solvent can be the composition (1). Further, the composition (1) can also be produced by mixing the fluorine-containing polymer (A) and the compound (BC).

The composition (1) may have an average transmittance of 85% or more, 86% or more, 87% or more, 85 to 95%, or 86 to 95%, in a wavelength range of 240 nm to 340 nm. A higher average transmittance is better. In the present specification, the average transmittance is determined as follows.

### Average Transmittance

The average transmittance of a sample at a predetermined wavelength (e.g., 240 nm to 340 nm) is measured using a Hitachi U-4100 spectrophotometer. The sample is a flat plate with an average thickness of 1 mm. An integrating sphere detector is used as the detector.

### Lens for Ultraviolet-Light-Emitting Elements

An embodiment of the present disclosure is a lens for ultraviolet-light-emitting elements, the lens comprising a fluorine-containing polymer (A), wherein the fluorine-containing polymer (A) comprises as a main component a structural unit represented by formula (A):

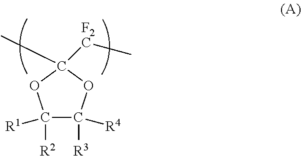

wherein R¹ to R⁴ each independently represent a fluorine atom, a C1-C5 perfluoroalkyl group, or a C1-C5 perfluoroalkoxy group, and has an average transmittance of 85% or more in a wavelength range of 240 nm to 340 nm.

The fluorine-containing polymer (A) has an average transmittance of as high as 85% or more in a wavelength range of 240 nm to 340 nm. The fluorine-containing polymer (A) is preferably produced by the production method of the present disclosure. According to the production method of the present disclosure, the fluorine-containing polymer (A) to be produced has a very low content of the compound (BC). Therefore, it is presumed that the average transmittance of the fluorine-containing polymer (A) in a wavelength range of 240 nm to 340 nm becomes high.

The average transmittance of the fluorine-containing polymer (A) in a wavelength range of 240 nm to 340 nm can be 85% or more, 86% or more, 87% or more, 85 to 95%, or 86 to 95%.

The lens for ultraviolet-light-emitting elements is desired to have high ultraviolet light permeability, and to be resistant to scratches and cracks. The lens for ultraviolet-light-emitting elements of the present disclosure is scratch-resistant due to the high surface hardness of the starting resin (fluorine-containing polymer (A)). The lens for ultraviolet-light-emitting elements of the present disclosure is crack-resistant due to the high toughness of the starting resin. Therefore, even when the starting resin is molded into a lens with a fine structure, the fine structure is less likely to crack or be scratched. For example, the lens for ultraviolet-light-emitting elements of the present disclosure is suitable for lenses with a fine structure, such as Fresnel lenses.

Examples of the lens for ultraviolet-light-emitting elements include Fresnel lenses, microlenses, curved lenses, non-curved lenses, biconvex lenses, plano-convex lenses, convex meniscus lenses, biconcave lenses, plano-concave lenses, concave meniscus lenses, cylindrical lenses, toroidal lenses, parabolic lenses, aspheric lenses, diffractive lenses, and their array lenses; and preferably Fresnel lenses, microlenses, biconvex lenses, plano-convex lenses, biconcave lenses, plano-concave lenses, aspheric lenses, diffractive lenses, and their array lenses.

The lens for ultraviolet-light-emitting elements can be produced by molding the fluorine-containing polymer (A) by a known lens molding method. The molding conditions can be adjusted appropriately from known conditions as needed. For example, it is desirable to produce a lens for ultraviolet-light-emitting elements by molding the fluorine-containing polymer (A) into a desired shape by injection molding in which the polymer is melted and injected. The fluorine-containing polymer (A) may be molded into a desired shape by other methods, for example, a press molding method in which the fluorine-containing polymer (A) is processed into a flat plate shape and pressed against a heated mold to form a lens for ultraviolet-light-emitting elements.

The ultraviolet light-emitting element to which the lens for ultraviolet-light-emitting elements is applied is preferably an ultraviolet light-emitting diode (LED). The ultraviolet light-emitting element mainly emits ultraviolet light, for example, light with a wavelength of 100 to 400 nm (preferably 240 to 340 nm, and more preferably 250 to 270 nm). The wavelengths include UV-A, UV-B, UV-C, etc., which are of high industrial value. The lens for ultraviolet-light-emitting elements can be applied to any type of ultraviolet light-emitting element; however, lenses applicable to UV-C light-emitting elements are preferred.

In the present specification, the “lens” does not include so-called “sealing materials” for light-emitting elements.

### Composition (2)

An embodiment of the present disclosure is a composition comprising a fluorine-containing polymer (A1) produced as a precipitate by polymerizing a fluorine-containing monomer (M1) represented by formula (M1):

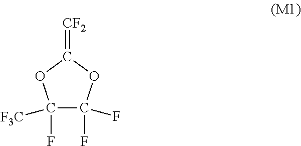

in the presence of the fluorine-containing monomer (M1), a polymerization initiator (B), and a solvent (D) (which may be referred to as “the composition (2)” in the present specification).

The fluorine-containing polymer (A1) comprises as a main component a structural unit represented by formula (A1):

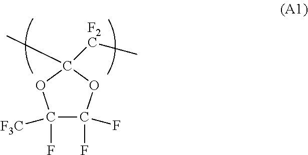

In the composition (2), the fluorine-containing monomer (M1) contains at least one compound selected from the group consisting of
a compound represented by formula (N1):

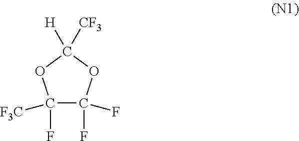 [SMILES: FC(F)(F)C1OC(F)(F)C(F)(C(F)(F)F)O1]

and
a compound represented by formula (N2):

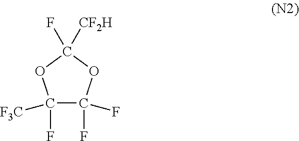 [SMILES: FC(F)C1(F)OC(F)(F)C(F)(C(F)(F)F)O1]

The content thereof is 0.0001 to 10 mass % based on the mass of a starting monomer, and can be 0.0001 to 8 mass % or 0.0001 to 6 mass %. The compounds (N1) and (N2) may be used singly or in a combination of two or more.

In the composition (2), the value (R_{A}) of the solubility index R, calculated based on the Hansen solubility parameters of the solvent (D) and the Hansen solubility parameters of the fluorine-containing polymer (A1), may be 4 or more.

In the composition (2), when the mass percentage of fluorine atoms in the polymerization initiator (B) is less than 50 mass %, the value (R_{B}) of the solubility index R, calculated based on the HSPs of the solvent (D) and the HSPs of the polymerization initiator (B), may be 9 or less, and preferably 8 or less.

In the composition (2), when the mass percentage of fluorine atoms in the polymerization initiator (B) is 50 mass % or more, the value (R_{B}) of the solubility index R, calculated based on the HSPs of the solvent (D) and the HSPs of the polymerization initiator (B), may be 6 or less, and preferably 5 or less.

In the composition (2), when the mass percentage of fluorine atoms in the decomposed product (C) is less than 50 mass %, the value (R_{C}) of the solubility index R, calculated based on the HSPs of the solvent (D) and the HSPs of the decomposed product (C), may be 9 or less, and preferably 8 or less.

In the composition (2), when the mass percentage of fluorine atoms in the decomposed product (C) is 50 mass % or more, the value (R_{C}) of the solubility index R, calculated based on the HSPs of the solvent (D) and the HSPs of the decomposed product (C), may be 6 or less, and preferably 5 or less.

Since the composition (2) has a low content of the compound (BC), the composition (2) is useful as a source of the fluorine-containing polymer (A1) with high ultraviolet light permeability (e.g., a source of the fluorine-containing polymer (A) used in products through which ultraviolet light passes (preferably lenses for ultraviolet-light-emitting elements)).

The fluorine-containing polymer (A1) is preferably produced by the method for producing a fluorine-containing polymer (A) of the present disclosure because the content of a polymerization initiator and/or its decomposed product becomes low. The fluorine-containing polymer (A1) may also be produced by a known production method and purified so that the content of a polymerization initiator and/or its decomposed product becomes low. As such a known production method, for example, it can be synthesized by polymerizing monomers corresponding to the structural units of the fluorine-containing polymer (A1). The polymerization method may be, for example, radical polymerization, bulk polymerization, precipitation polymerization, solution polymerization, suspension polymerization, or emulsion polymerization. A preferred known production method is precipitation polymerization, in terms of the high permeability of the fluorine-containing polymer (A1).

The content of the fluorine-containing polymer (A1) in the composition (2) can be 95 mass % or more based on the mass of the composition (2). The content can be preferably 97 to 100 mass %, more preferably 98 to 100 mass %, and particularly preferably 99 to 100 mass %.

Regarding the composition (2), the details of the polymerization are applicable to the details described above regarding the polymerization step.

The composition (2) may contain other components in addition to the fluorine-containing polymer (A1), but preferably does not contain other components. Examples of other components include metals such as Fe and Ni, the residual solvent, and the like. The content of other components in the composition (2) may be 0 to 0.1 mass % or 0 to 0.01 mass %.

The composition (2) can be produced by the production method of the present disclosure. For example, the solid obtained in the filtration step or a washed product obtained by washing the solid with a solvent can be the composition (2).

Although embodiments are described above, it can be understood that various modifications in form and details may be made without departing from the spirit and scope of the claims.

The present disclosure includes, for example, the following.

### Item 1.

A composition comprising:
a fluorine-containing polymer (A); and
at least one compound (BC) selected from the group consisting of a polymerization initiator (B) and a decomposed product (C) of the polymerization initiator (B),
wherein the compound (BC) is contained in an amount of 0.1 to 100 ppm by mass based on the mass of the composition, and
the fluorine-containing polymer (A) comprises as a main component a structural unit represented by formula (A):

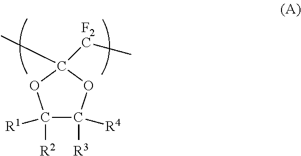

wherein R¹ to R⁴ each independently represent a fluorine atom, a C1-C5 perfluoroalkyl group, or a C1-C5 perfluoroalkoxy group.

### Item 2.

The composition according to Item 1, wherein the polymerization initiator (B) is a peroxide.

### Item 3.

The composition according to Item 1, wherein the polymerization initiator (B) is at least one compound selected from the group consisting of
a compound represented by formula (B1):

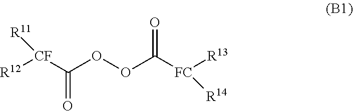

wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula (B2):

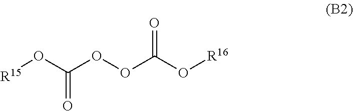

wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3):

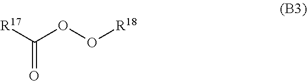

wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

### Item 4.

The composition according to any one of Items 1 to 3, wherein the decomposed product (C) is at least one compound selected from the group consisting of
a compound represented by formula (C1):

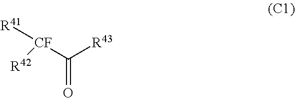

wherein R⁴¹ and R⁴² each independently represent a fluorine atom, a C1-C20 alkyl group, or a C1-C20 fluoroalkyl group, the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula (C2):

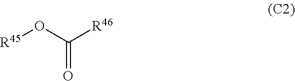

wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and
a compound represented by formula (C3):

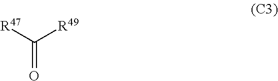

wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

### Item 5.

The composition according to any one of Items 1 to 4, which has an average transmittance of 85% or more in a wavelength range of 240 nm to 340 nm.

### Item 6.

The composition according to any one of Items 1 to 5, wherein the structural unit represented by formula (A) is a structural unit represented by formula (A1):

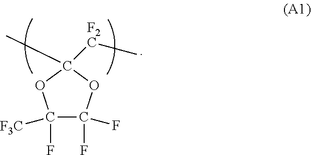

### Item 7.

The composition according to any one of Items 1 and 4 to 6, wherein the polymerization initiator (B) is at least one compound selected from the group consisting of
a compound represented by formula (B1):

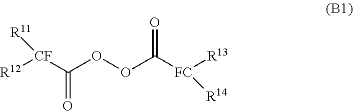

wherein R¹¹ and R¹³ each represent a fluorine atom, and R¹² and R¹⁴ each independently represent
—(CF₂)ₙ₁CF₃ wherein n1 represents an integer of 0 to 5,
(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5; a compound represented by formula (B2):

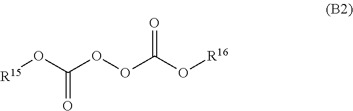

wherein R¹⁵ and R¹⁶ each independently represent a C1-C7 linear or branched alkyl group, or a C1-C7 linear or branched fluoroalkyl group; and
a compound represented by formula (B3):

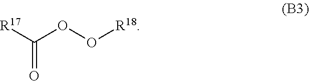

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

### Item 8.

The composition according to any one of Items 1 to 3 and 5 to 7, wherein the decomposed product (C) is at least one compound selected from the group consisting of a compound represented by formula (C1):

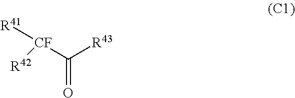

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2):

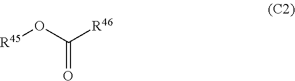

wherein R⁴⁵ represents a C1-C7 linear or branched alkyl group, or a C1-C7 linear or branched fluoroalkyl group, and R⁴⁶ represents a fluorine atom or a hydroxy group; and a compound represented by formula (C3):

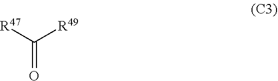

wherein R⁴⁷ represents methyl, tert-butyl, tert-nonyl, trifluoromethyl, perfluoroisopentyl, or phenyl, and R⁴⁹ represents a fluorine atom or a hydroxy group.

### Item 9.

The composition according to Item 1 or 5,
wherein the structural unit represented by formula (A) is a structural unit represented by formula (A1):

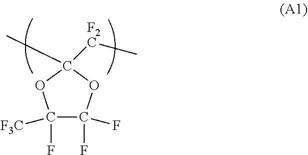

the polymerization initiator (B) is at least one compound selected from the group consisting of a compound represented by formula (B1):

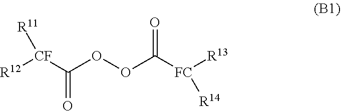

wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula (B2):

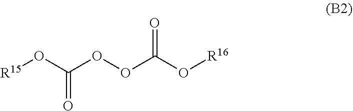

wherein R¹⁵ and R¹⁶ each independently represent a C1-C7 linear or branched alkyl group, or a C1-C7 linear or branched fluoroalkyl group, and
a compound represented by formula (B3):

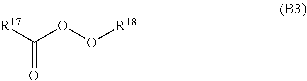

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl; and
the decomposed product (C) is at least one compound selected from the group consisting of
   a compound represented by formula (C1):

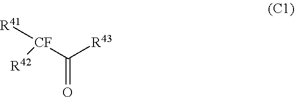

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2):

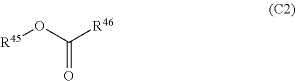

wherein R⁴⁵ represents a C1-C7 linear or branched alkyl group, or a C1-C7 linear or branched fluoroalkyl group, and R⁴⁶ represents a fluorine atom or a hydroxy group, and
a compound represented by formula (C3):

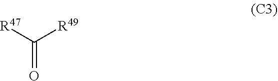

wherein R⁴⁷ represents methyl, tert-butyl, tert-nonyl, trifluoromethyl, perfluoroisopentyl, or phenyl, and R⁴⁹ represents a fluorine atom or a hydroxy group.

### Item 10.

A method for producing a fluorine-containing polymer (A), comprising
a polymerization step of polymerizing a fluorine-containing monomer (M) represented by formula (M):

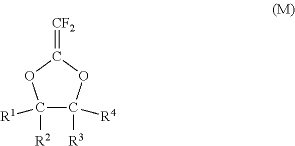

wherein R¹ to R⁴ each independently represent a fluorine atom, a C1-C5 perfluoroalkyl group, or a C1-C5 perfluoroalkoxy group in the presence of the fluorine-containing monomer (M), a polymerization initiator (B), and a solvent (D) to produce a fluorine-containing polymer (A) as a precipitate,
wherein the fluorine-containing polymer (A) comprises as a main component a structural unit represented by formula (A):

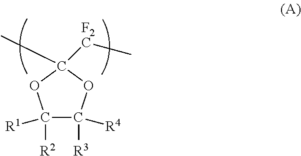

wherein R¹ to R⁴ are as defined above,
a value (R_{A}) of a solubility index R, calculated based on the Hansen solubility parameters of the solvent (D) and the Hansen solubility parameters of the fluorine-containing polymer (A), is 4 or more,
a value (R_{B}) of a solubility index R, calculated based on the Hansen solubility parameters of the solvent (D) and the Hansen solubility parameters of the polymerization initiator (B), is 9 or less when the mass percentage of fluorine atoms in the polymerization initiator (B) is less than 50 mass % and is 6 or less when the same mass percentage is 50 mass % or more,
a value (R_{C}) of a solubility index R, calculated based on the Hansen solubility parameters of the solvent (D) and the Hansen solubility parameters of the decomposed product (C) of the polymerization initiator (B), is 9 or less when the mass percentage of fluorine atoms in the decomposed product (C) is less than 50 mass % and is 6 or less when the same mass percentage is 50 mass % or more, and
the solubility index R is calculated according to the following equation:

$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$

wherein δD₁, δP₁, and δH₁ respectively represent a dispersion parameter, a polarity parameter, and a hydrogen bonding parameter in the Hansen solubility parameters of element 1, and δD₂, δP₂, and δH₂ respectively represent a dispersion parameter, a polarity parameter, and a hydrogen bonding parameter in the Hansen solubility parameters of element 2.

### Item 11.

The method for producing a fluorine-containing polymer (A) according to Item 10, further comprising a filtration step of filtering the precipitate and a polymerization liquid produced in the polymerization step to remove the polymerization liquid to thereby obtain the fluorine-containing polymer (A).

### Item 12.

The method for producing a fluorine-containing polymer (A) according to Item 10 or 11, wherein the polymerization initiator (B) is a peroxide.

### Item 13.

The method for producing a fluorine-containing polymer (A) according to Item 10 or 11, wherein the polymerization initiator (B) is at least one compound selected from the group consisting of
a compound represented by formula (B1):

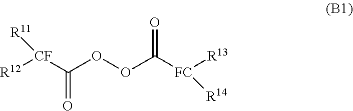

wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula (B2):

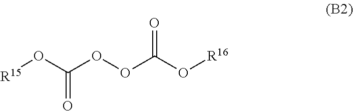

wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3):

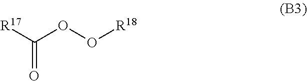

wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

### Item 14.

The method for producing a fluorine-containing polymer (A) according to any one of Items 10 to 13, wherein the decomposed product (C) of the polymerization initiator (B) is at least one compound selected from the group consisting of a compound represented by formula (C1):

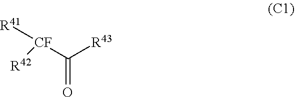

wherein R⁴¹ and R⁴² each independently represent a fluorine atom, a C1-C20 alkyl group, or a C1-C20 fluoroalkyl group,
the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula (C2):

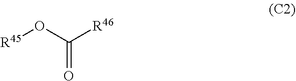

wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and
a compound represented by formula (C3):

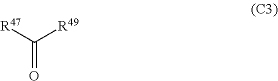

wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

### Item 15.

The method for producing a fluorine-containing polymer (A) according to any one of Items 10 to 14,
wherein the fluorine-containing monomer (M) is a compound represented by formula (M1):

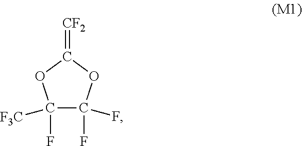

and
the structural unit represented by formula (A) is a structural unit represented by formula (A1):

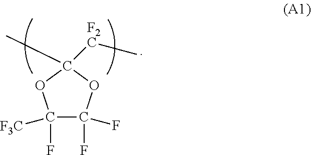

### Item 16.

The method for producing a fluorine-containing polymer (A) according to any one of Items 10 to 15,
wherein the fluorine-containing monomer (M) contains a compound (N) in an amount of 0.0001 to 10 mass % based on the mass of a starting monomer, and
the compound (N) is a compound represented by formula (N):

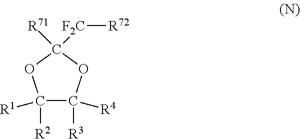

wherein R¹ to R⁴ each independently represent a fluorine atom, a C1-C5 perfluoroalkyl group, or a C1-C5 perfluoroalkoxy group, R⁷¹ and R⁷² each independently represent a hydrogen atom or a fluorine atom, and when one of R⁷¹ or R⁷² is a hydrogen atom, the other is a fluorine atom.

### Item 17.

The method for producing a fluorine-containing polymer (A) according to Item 16, wherein the compound (N) is at least one compound selected from the group consisting of
a compound represented by formula (N1):

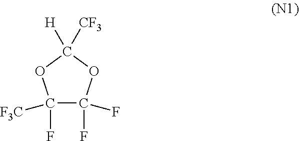 [SMILES: FC(F)(F)C1OC(F)(F)C(F)(C(F)(F)F)O1]

and
a compound represented by formula (N2):

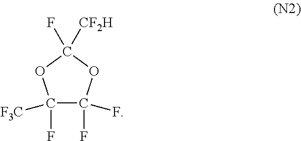 [SMILES: FC(F)C1(F)OC(F)(F)C(F)(C(F)(F)F)O1]

### Item 18.

The method for producing a fluorine-containing polymer (A) according to any one of Items 10, 11, and 14 to 17, wherein the polymerization initiator (B) is at least one compound selected from the group consisting of
a compound represented by formula (B1):

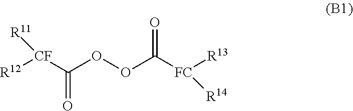

wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5; a compound represented by formula (B2):

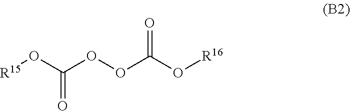

wherein R¹⁵ and R¹⁶ each independently represent a C1-C7 linear or branched alkyl group, or a C1-C7 linear or branched fluoroalkyl group; and
a compound represented by formula (B3):

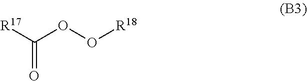

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

### Item 19.

The method for producing a fluorine-containing polymer (A) according to any one of Items 10 to 13 and 15 to 18, wherein the decomposed product (C) is at least one compound selected from the group consisting of
a compound represented by formula (C1):

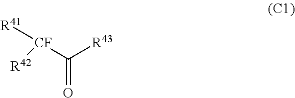

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2):

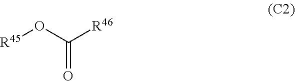

wherein R⁴⁵ represents a C1-C7 linear or branched alkyl group, or a C1-C7 linear or branched fluoroalkyl group, and R⁴⁶ represents a fluorine atom or a hydroxy group; and a compound represented by formula (C3):

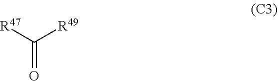

wherein R⁴⁷ represents methyl, tert-butyl, tert-nonyl, trifluoromethyl, perfluoroisopentyl, or phenyl, and R⁴⁹ represents a fluorine atom or a hydroxy group.

### Item 20.

The method for producing a fluorine-containing polymer (A) according to any one of Items 10 to 19, wherein the solvent (D) is at least one selected from the group consisting of hydrofluoroethers and fluorine-containing alcohols.

### Item 21.

The method for producing a fluorine-containing polymer (A) according to Item 10 or 11,
wherein the fluorine-containing monomer (M) is a compound represented by formula (M1):

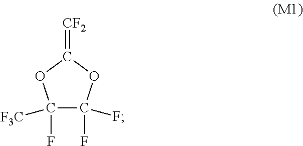

the fluorine-containing monomer (M) contains a compound (N) in an amount of 0.0001 to 10 mass % based on the mass of a starting monomer;
the compound (N) is at least one compound selected from the group consisting of
a compound represented by formula (N1):

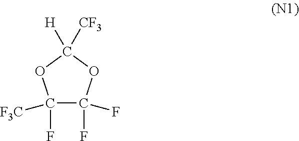 [SMILES: FC(F)(F)C1OC(F)(F)C(F)(C(F)(F)F)O1]

and
a compound represented by formula (N2),

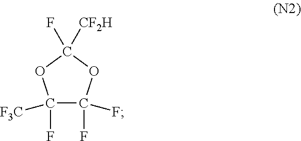 [SMILES: FC(F)C1(F)OC(F)(F)C(F)(C(F)(F)F)O1]

the structural unit represented by formula (A) is a structural unit represented by formula (A1):

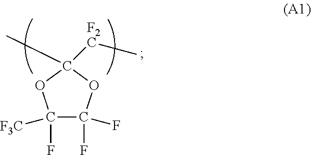

the polymerization initiator (B) is at least one compound selected from the group consisting of
   a compound represented by formula (B1):

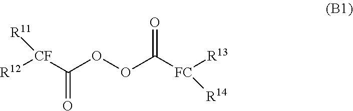

wherein R¹¹ and R¹³ each represent a fluorine atom, and R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula (B2):

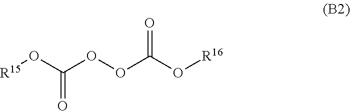

wherein R¹⁵ and R¹⁶ each independently represent a C1-C7 linear or branched alkyl group, or a C1-C7 linear or branched fluoroalkyl group, and
a compound represented by formula (B3):

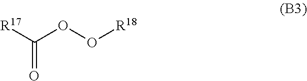

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl;
the decomposed product (C) is at least one compound selected from the group consisting of
   a compound represented by formula (C1):

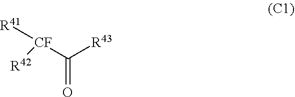

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2):

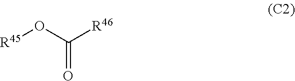

wherein R⁴⁵ represents a C1-C7 linear or branched alkyl group, or a C1-C7 linear or branched fluoroalkyl group, and R⁴⁶ represents a fluorine atom or a hydroxy group, and
   a compound represented by formula (C3):

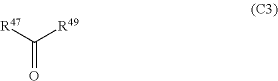

wherein R⁴⁷ represents methyl, tert-butyl, tert-nonyl, trifluoromethyl, perfluoroisopentyl, or phenyl, and R⁴⁹ represents a fluorine atom or a hydroxy group; and
the solvent (D) is at least one selected from the group consisting of hydrofluoroethers and fluorine-containing alcohols.

### Item 22.

A lens for ultraviolet-light-emitting elements, the lens comprising a fluorine-containing polymer (A),
wherein the fluorine-containing polymer (A) comprises as a main component a structural unit represented by formula (A):

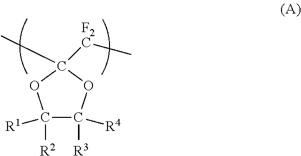

wherein R¹ to R⁴ each independently represent a fluorine atom, a C1-C5 perfluoroalkyl group, or a C1-C5 perfluoroalkoxy group, and
the fluorine-containing polymer (A) has an average transmittance of 85% or more in a wavelength range of 240 nm to 340 nm.

### Item 23.

The lens for ultraviolet-light-emitting elements according to Item 22, wherein the structural unit represented by formula (A) is a structural unit represented by formula (A1):

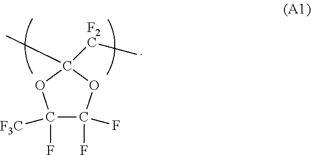

### Item 24.

A composition comprising a fluorine-containing polymer (A1) produced as a precipitate by polymerizing a fluorine-containing monomer (M1) represented by formula (M1):

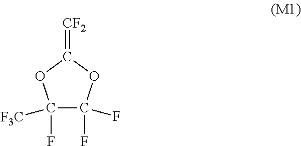

in the presence of the fluorine-containing monomer (M1), a polymerization initiator (B), and a solvent (D),
   wherein the fluorine-containing polymer (A1) comprises as a main component a structural unit represented by formula (A1):

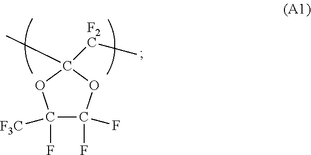

the fluorine-containing monomer (M1) contains at least one compound selected from the group consisting of a compound represented by formula (N1):

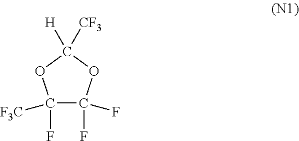 [SMILES: FC(F)(F)C1OC(F)(F)C(F)(C(F)(F)F)O1]

and
a compound represented by formula (N2):

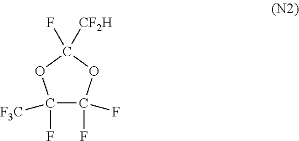 [SMILES: FC(F)C1(F)OC(F)(F)C(F)(C(F)(F)F)O1]

in an amount of 0.0001 to 10 mass % based on the mass of a starting monomer;
   a value (R_{A1}) of a solubility index R, calculated based on the Hansen solubility parameters of the solvent (D) and the Hansen solubility parameters of the fluorine-containing polymer (A1), is 4 or more;
   a value (R_{B}) of a solubility index R, calculated based on the Hansen solubility parameters of the solvent (D) and the Hansen solubility parameters of the polymerization initiator (B), is 9 or less when the mass percentage of fluorine atoms in the polymerization initiator (B) is less than 50 mass %, and is 6 or less when the same mass percentage is 50 mass % or more;
   a value (R_{C}) of a solubility index R, calculated based on the Hansen solubility parameters of the solvent (D) and the Hansen solubility parameters of the decomposed product (C) of the polymerization initiator (B), is 9 or less when the mass percentage of fluorine atoms in the decomposed product (C) is less than 50 mass %, and is 6 or less when the same mass percentage is 50 mass % or more; and
   the solubility index R is calculated according to the following equation:

$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$

wherein δD₂, δP₂, and δH₁ respectively represent a dispersion parameter, a polarity parameter, and a hydrogen bonding parameter in the Hansen solubility parameters of element 1, and δD₂, δP₂, and δH₂ respectively represent a dispersion parameter, a polarity parameter, and a hydrogen bonding parameter in the Hansen solubility parameters of element 2.

### Item 25.

The composition comprising a fluorine-containing polymer (A1) according to Item 24, wherein the polymerization initiator (B) is a peroxide.

### Item 26.

The composition comprising a fluorine-containing polymer (A1) according to Item 24, wherein the polymerization initiator (B) is at least one compound selected from the group consisting of
a compound represented by formula (B1):

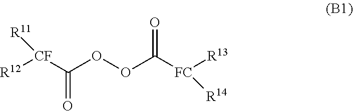

wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula (B2):

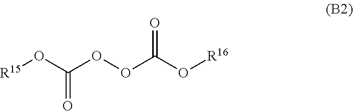

wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3):

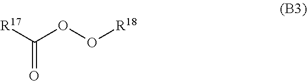

wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

### Item 27.

The composition comprising a fluorine-containing polymer (A1) according to Item 24 or 25, wherein the decomposed product (C) of the polymerization initiator (B) is at least one compound selected from the group consisting of a compound represented by formula (C1):

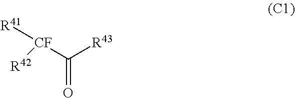

wherein R⁴¹ and R⁴² each independently represent a fluorine atom, a C1-C20 alkyl group, or a C1-C20 fluoroalkyl group,
the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula (C2):

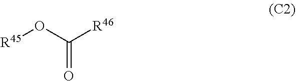

wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and a compound represented by formula (C3):

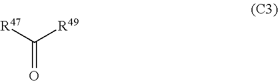

wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

### Item 28.

The composition comprising a fluorine-containing polymer (A1) according to Item 24, wherein the polymerization initiator (B) is at least one compound selected from the group consisting of
a compound represented by formula (B1):

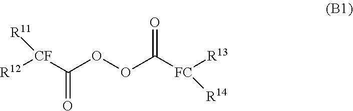

wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5;
a compound represented by formula (B2):

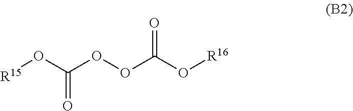

wherein R¹⁵ and R¹⁶ each independently represent a C1-C7 linear or branched alkyl group, or a C1-C7 linear or branched fluoroalkyl group; and
a compound represented by formula (B3):

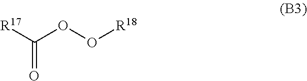

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

### Item 29.

The composition comprising a fluorine-containing polymer (A1) according to any one of Items 24 to 26 and 28, wherein the decomposed product (C) is at least one compound selected from the group consisting of
a compound represented by formula (C1):

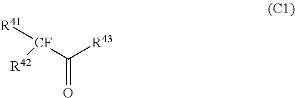

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2):

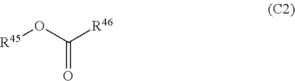

wherein R⁴⁵ represents a C1-C7 linear or branched alkyl group, or a C1-C7 linear or branched fluoroalkyl group, and R⁴⁶ represents a fluorine atom or a hydroxy group; and
a compound represented by formula (C3):

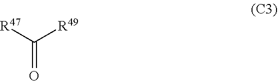

wherein R⁴⁷ represents methyl, tert-butyl, tert-nonyl, trifluoromethyl, perfluoroisopentyl, or phenyl, and R⁴⁹ represents a fluorine atom or a hydroxy group.

### Item 30.

The composition comprising a fluorine-containing polymer (A1) according to any one of Items 24 to 29, wherein the solvent (D) is at least one selected from the group consisting of hydrofluoroethers and fluorine-containing alcohols.

### Item 31.

The composition comprising a fluorine-containing polymer (A1) according to Item 24,
wherein the polymerization initiator (B) is at least one compound selected from the group consisting of
   a compound represented by formula (B1):

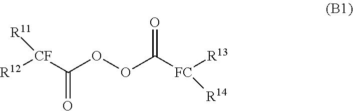

wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula (B2):

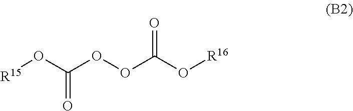

wherein R¹⁵ and R¹⁶ each independently represent a C1-C7 linear or branched alkyl group, or a C1-C7 linear or branched fluoroalkyl group, and
a compound represented by formula (B3):

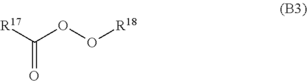

wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl;
the decomposed product (C) is at least one compound selected from the group consisting of
   a compound represented by formula (C1):

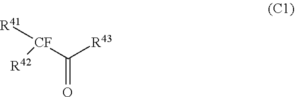

wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2):

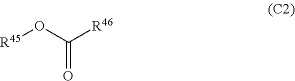

wherein R⁴⁵ represents a C1-C7 linear or branched alkyl group, or a C1-C7 linear or branched fluoroalkyl group, and R⁴⁶ represents a fluorine atom or a hydroxy group, and a compound represented by formula (C3):

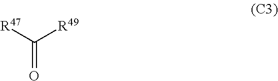

wherein R⁴⁷ represents methyl, tert-butyl, tert-nonyl, trifluoromethyl, perfluoroisopentyl, or phenyl, and R⁴⁹ represents a fluorine atom or a hydroxy group; and
the solvent (D) is at least one selected from the group consisting of hydrofluoroethers and fluorine-containing alcohols.

### EXAMPLES

An embodiment of the present disclosure is described in more detail below with reference to Examples; however, the present disclosure is not limited to these.

The symbols and abbreviations in the Examples are used with the following meanings.
PFMMD: fluorine-containing monomer (M1)
HF adduct: compound (N1)
NPP: di-n-propyl peroxydicarbonate 50% methanol solution (produced by NOF Corporation)
DHP: bis(2,2,3,3,4,4,5,5,6,6,7,7-dodecafluoro-1-oxoheptyl)peroxide 8% diluted with fluorine-based solvent (produced by NOF Corporation)
TBPOA: tert-butyl peroxyacetate 50% diluted with hydrocarbon solvent (produced by NOF Corporation)
HFE-356mec: 1,1,2,3,3,3-hexafluoropropyl methyl ether
n=2 Alcohol: 2,2,3,3,4,4,5,5-octafluoro-1-pentanol (produced by Daikin Industries, Ltd.)
FC-3283: perfluoro-n-tripropylamine (produced by 3M)

The polymerization initiators and their decomposed products are as shown below. In the Examples, the decomposed product of NPP was a —COOH compound, the decomposed products of DHP were —COOH and —COF compounds, and the decomposed product of TBPOA was a —COOH compound.

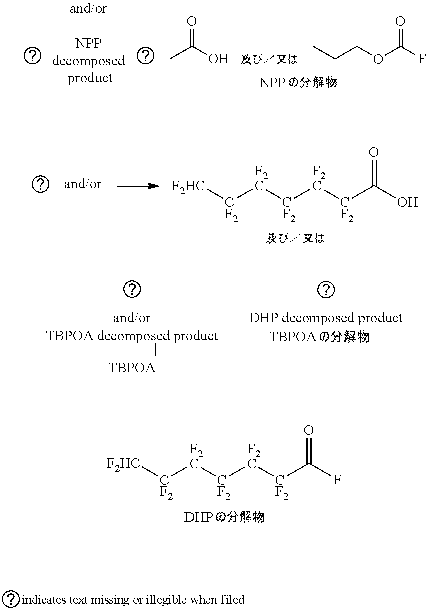

The Hansen solubility parameters are as shown below.

TABLE 1

| HSP | | |
|---|---|---|
| δD | δP | δH |

Solvents

| | | | |
|---|---|---|---|
| HFE-356mec | 12.8 | 3.0 | 2.0 |
| n = 2 Alcohol | 14.8 | 5.4 | 9.7 |
| CF3CH2OH | 15.4 | 8.3 | 16.4 |
| CHCl3 | 17.8 | 3.1 | 5.7 |
| FC-3283 | 12.2 | 1.1 | 0.5 |

Polymerization initiators

| | | | |
|---|---|---|---|
| NPP | 15.8 | 8.5 | 6.0 |
| DHP | 12.9 | 3.5 | 3.9 |
| TBPOA | 15.5 | 5.8 | 9.5 |

Decomposed products of polymerization initiators

| | | | |
|---|---|---|---|
| NPP decomposed | 16.3 | 9.2 | 7.2 |
| product (—COOH) | | | |
| DHP decomposed | 14.4 | 4.5 | 7.8 |
| product (—COOH) | | | |
| DHP decomposed | 12.9 | 2.1 | 2.1 |
| product (—COF) | | | |
| TBPOA decomposed | 14.5 | 8.0 | 13.5 |
| product (—COOH) | | | |

Fluorine-containing polymer

| | | | |
|---|---|---|---|
| Polymer A1 | 11.2 | 3.9 | 1.6 |

### Average Transmittance

The average transmittance of a sample at a predetermined wavelength (e.g., 240 nm to 340 nm) was measured using a Hitachi U-4100 spectrophotometer. The sample was a flat plate with an average thickness of 1 mm. An integrating sphere detector was used as the detector.

### Example 1

In a 300 mL stainless steel autoclave, 100 g of the fluorine-containing monomer (M1) containing 3.6% of the compound (N1), and 400 g of HFE-356mec (solvent) were added, followed by 0.1 g of DHP (polymerization initiator). After the autoclave was cooled to −78° C., the atmosphere in the system was replaced with nitrogen gas, and the system was filled with nitrogen gas until the internal pressure reached 0.1 MPaG. The mixture was subjected to a polymerization reaction at 15° C. for 24 hours, and the resulting fluorine-containing polymer (A1) was precipitated in the solvent to give a polymer-solvent mixture. The mixture was filtered and dried at 120° C. to obtain a solid polymer. The polymer corresponded to the composition (1) and also to the composition (2).

1 g of the polymer was dissolved in 10 g of hexafluorobenzene, and the concentration of the compound (BC) in the polymer was quantified by gas chromatography to be 20 ppm.

The polymer was melt-molded to produce a flat plate with an average thickness of 1 mm. The average transmittance (240 nm to 340 nm) of the flat plate was 90%.

A Fresnel lens was produced by cutting a flat plate with an average thickness of 1 mm, which was molded in the same manner. When the uneven structure of the Fresnel lens surface was observed using a VKX-1000 laser microscope produced by Keyence Corporation, the surface roughness Sa was 74 nm and Sq was 118 nm.

### Example 2

The same procedure as in Example 1 was carried out, except that NPP was used in place of DHP and the polymerization temperature was changed to 40° C. The concentration of the compound (BC) in the resulting polymer was 43 ppm, and the average transmittance (240 nm to 340 nm) of the flat plate was 87%.

### Example 3

The same procedure as in Example 1 was carried out, except that n=2 Alcohol was used in place of HFE-356mec. The concentration of the compound (BC) in the resulting polymer was 17 ppm, and the average transmittance (240 nm to 340 nm) of the flat plate was 92%.

### Example 4

The same procedure as in Example 1 was carried out, except that n=2 Alcohol was used in place of HFE-356mec, TBPOA was used in place of DHP, and the polymerization temperature was changed to 100° C. The concentration of the compound (BC) in the resulting polymer was 23 ppm, and the average transmittance (240 nm to 340 nm) of the flat plate was 90%.

### Comparative Example 1

The same procedure as in Example 1 was carried out, except that CF₃CH₂OH was used in place of HFE-356mec. The concentration of the compound (BC) in the resulting polymer was 163 ppm, and the average transmittance (240 nm to 340 nm) of the flat plate was 81%.

### Comparative Example 2

The same procedure as in Example 1 was carried out, except that CF₃CH₂OH was used in place of HFE-356mec, NPP was used in place of DHP, and the polymerization temperature was changed to 40° C. The concentration of the compound (BC) in the resulting polymer was 155 ppm, and the average transmittance (240 nm to 340 nm) of the flat plate was 83%.

### Comparative Example 3

The same procedure as in Example 1 was carried out, except that CHCl₃ was used in place of HFE-356mec. The concentration of the compound (BC) in the resulting polymer was 124 ppm, and the average transmittance (240 nm to 340 nm) of the flat plate was 80%.

### Comparative Example 4

The same procedure as in Example 1 was carried out, except that the fluorine-containing monomer (M1) containing 17% of the compound (N1) was used in place of the fluorine-containing monomer (M1) containing 3.6% of the compound (N1), n=2 Alcohol was used in place of HFE-356mec, TBPOA was used in place of DHP, and the polymerization temperature was changed to 100° C. The concentration of the compound (BC) in the resulting polymer was 118 ppm, and the average transmittance (240 nm to 340 nm) of the flat plate was 83%.

### Comparative Example 5

The same procedure as in Example 1 was carried out, except that FC-3283 was used in place of HFE-356mec. The mixture after polymerization was a solution in which the produced polymer (A1) was dissolved in the solvent.

The various conditions, solubility index, measurement results, etc. of the Examples and Comparative Examples are summarized in Table 2 below.

TABLE 2

| | | | | | Mass | |
|---|---|---|---|---|---|---|
| | | | | | percentage of | |
| | | | | | F atoms in | |
| | | Compound | | Polymerization | decomposed | R_{A} |
| | Monomer | (N1) | Solvent | initiator | product | (polymer) |
| Ex. 1 | PFMMD | 3.6% | HFE-356mec | DHP | 66% | 4.7 |
| Ex. 2 | PFMMD | 3.6% | HFE-356mec | NPP | 0% | 4.7 |
| Ex. 3 | PFMMD | 3.6% | n = 2 Alcohol | DHP | 66% | 10.9 |
| Ex. 4 | PFMMD | 3.6% | n = 2 Alcohol | TBPOA | 0% | 10.9 |
| Comp. Ex. 1 | PFMMD | 3.6% | CF3CH2OH | DHP | 66% | 17.5 |
| Comp. Ex. 2 | PFMMD | 3.6% | CF3CH2OH | NPP | 0% | 17.5 |
| Comp. Ex. 3 | PFMMD | 3.6% | CHCl3 | DHP | 66% | 13.1 |
| Comp. Ex. 4 | PFMMD | 17% | n = 2 Alcohol | TBPOA | 0% | 10.9 |
| Comp. Ex. 5 | PFMMD | 3.6% | FC-3283 | DHP | 66% | 2.4 |

| | | | | | State of |
|---|---|---|---|---|---|
| | | | Concentration | | polymer in |
| | | R_{C} | (ppm) of | Average | polymerization |
| | R_{B} | (decomposed | compound | transmittance | reaction |
| | (initiator) | product) | (BC) | (%) | solution |
| Ex. 1 | 5.0 | 2.1 | 20 | 90% | Precipitated |
| Ex. 2 | 8.2 | 6.6 | 43 | 87% | Precipitated |
| Ex. 3 | 2.2 | 7.2 | 17 | 92% | Precipitated |
| Ex. 4 | 4.6 | 1.5 | 23 | 90% | Precipitated |
| Comp. Ex. 1 | 9.6 | 14.3 | 163 | 81% | Precipitated |
| Comp. Ex. 2 | 9.4 | 10.4 | 155 | 83% | Precipitated |
| Comp. Ex. 3 | 7.3 | 10.0 | 124 | 80% | Precipitated |
| Comp. Ex. 4 | 4.6 | 1.5 | 118 | 83% | Precipitated |
| Comp. Ex. 5 | 8.6 | 4.4 | No data | No data | Dissolved |

1. A composition comprising:
a fluorine-containing polymer (A); and
at least one compound (BC) selected from the group consisting of a polymerization initiator (B) and a decomposed product (C) of the polymerization initiator (B),
wherein the compound (BC) is contained in an amount of 0.1 to 100 ppm by mass based on the mass of the composition, and
the fluorine-containing polymer (A) comprises as a main component a structural unit represented by formula (A): 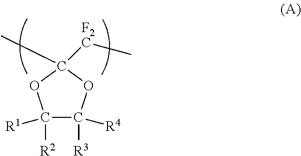
wherein R¹ to R⁴ each independently represent a fluorine atom, a C1-C5 perfluoroalkyl group, or a C1-C5 perfluoroalkoxy group.

2. The composition according to claim 1, wherein the polymerization initiator (B) is a peroxide.

3. The composition according to claim 1, wherein the polymerization initiator (B) is at least one compound selected from the group consisting of a compound represented by formula (B1): 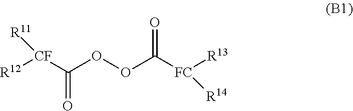
wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula (B2): 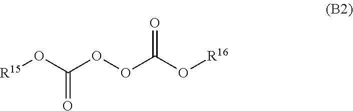
wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3): 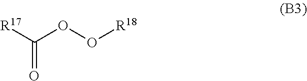
wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

4. The composition according to claim 1, wherein the decomposed product (C) is at least one compound selected from the group consisting of
a compound represented by formula (C1): 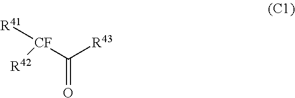
wherein R⁴¹ and R⁴² each independently represent a fluorine atom, a C1-C20 alkyl group, or a C1-C20 fluoroalkyl group,
the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula (C2): 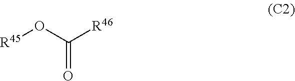
wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and
a compound represented by formula (C3): 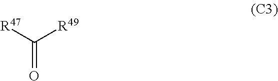
wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

5. The composition according to claim 1, which has an average transmittance of 85% or more in a wavelength range of 240 nm to 340 nm.

6. The composition according to claim 1, wherein the structural unit represented by formula (A) is a structural unit represented by formula (A1):

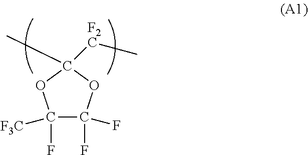

7. The composition according to claim 1, wherein the polymerization initiator (B) is at least one compound selected from the group consisting of
a compound represented by formula (B1): 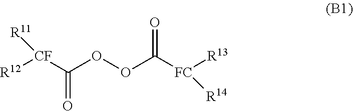
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5;
a compound represented by formula (B2): 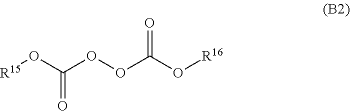
wherein R¹⁵ and R¹⁶ each independently represent a C1-C7 linear or branched alkyl group, or a C1-C7 linear or branched fluoroalkyl group; and
a compound represented by formula (B3): 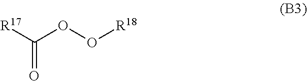
wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

8. The composition according to claim 1, wherein the decomposed product (C) is at least one compound selected from the group consisting of
a compound represented by formula (C1): 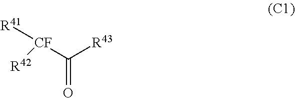
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2): 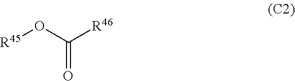
wherein R⁴⁵ represents a C1-C7 linear or branched alkyl group, or a C1-C7 linear or branched fluoroalkyl group, and R⁴⁶ represents a fluorine atom or a hydroxy group; and
a compound represented by formula (C3): 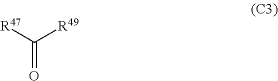
wherein R⁴⁷ represents methyl, tert-butyl, tert-nonyl, trifluoromethyl, perfluoroisopentyl, or phenyl, and R⁴⁹ represents a fluorine atom or a hydroxy group.

9. The composition according to claim 1,
wherein the structural unit represented by formula (A) is a structural unit represented by formula (A1): 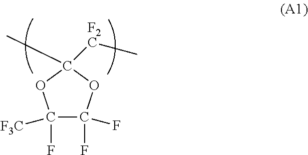
the polymerization initiator (B) is at least one compound selected from the group consisting of
a compound represented by formula (B1): 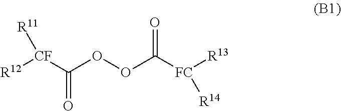
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula (B2): 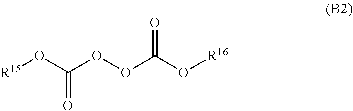
wherein R¹⁵ and R¹⁶ each independently represent a C1-C7 linear or branched alkyl group, or a C1-C7 linear or branched fluoroalkyl group, and
a compound represented by formula (B3): 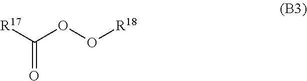
wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl; and
the decomposed product (C) is at least one compound selected from the group consisting of
a compound represented by formula (C1): 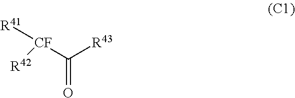
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2): 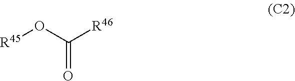
wherein R⁴⁵ represents a C1-C7 linear or branched alkyl group, or a C1-C7 linear or branched fluoroalkyl group, and R⁴⁶ represents a fluorine atom or a hydroxy group, and
a compound represented by formula (C3): 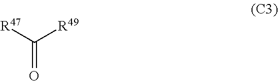
wherein R⁴⁷ represents methyl, tert-butyl, tert-nonyl, trifluoromethyl, perfluoroisopentyl, or phenyl, and R⁴⁹ represents a fluorine atom or a hydroxy group.

10. A method for producing a fluorine-containing polymer (A), comprising
a polymerization step of polymerizing a fluorine-containing monomer (M) represented by formula (M): 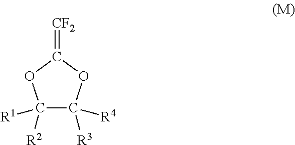
wherein R¹ to R⁴ each independently represent a fluorine atom, a C1-C5 perfluoroalkyl group, or a C1-C5 perfluoroalkoxy group
in the presence of the fluorine-containing monomer (M), a polymerization initiator (B), and a solvent (D) to produce a fluorine-containing polymer (A) as a precipitate,
wherein the fluorine-containing polymer (A) comprises as a main component a structural unit represented by formula (A): 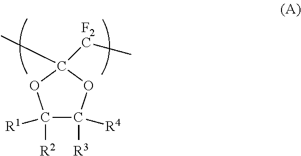
wherein R¹ to R⁴ are as defined above,
a value (R_{A}) of a solubility index R, calculated based on the Hansen solubility parameters of the solvent (D) and the Hansen solubility parameters of the fluorine-containing polymer (A), is 4 or more,
a value (R_{B}) of a solubility index R, calculated based on the Hansen solubility parameters of the solvent (D) and the Hansen solubility parameters of the polymerization initiator (B), is 9 or less when the mass percentage of fluorine atoms in the polymerization initiator (B) is less than 50 mass %, and is 6 or less when the same mass percentage is 50 mass % or more,
a value (R_{C}) of a solubility index R, calculated based on the Hansen solubility parameters of the solvent (D) and the Hansen solubility parameters of the decomposed product (C) of the polymerization initiator (B), is 9 or less when the mass percentage of fluorine atoms in the decomposed product (C) is less than 50 mass %, and is 6 or less when the same mass percentage is 50 mass % or more, and
the solubility index R is calculated according to the following equation:
$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$
wherein δD₁, δP₁, and δH₁ respectively represent a dispersion parameter, a polarity parameter, and a hydrogen bonding parameter in the Hansen solubility parameters of element 1, and δD₂, δP₂, and δH₂ respectively represent a dispersion parameter, a polarity parameter, and a hydrogen bonding parameter in the Hansen solubility parameters of element 2.

11. The method for producing a fluorine-containing polymer (A) according to claim 10, further comprising a filtration step of filtering the precipitate and a polymerization liquid produced in the polymerization step to remove the polymerization liquid to thereby obtain the fluorine-containing polymer (A).

12. The method for producing a fluorine-containing polymer (A) according to claim 10, wherein the polymerization initiator (B) is a peroxide.

13. The method for producing a fluorine-containing polymer (A) according to claim 10, wherein the polymerization initiator (B) is at least one compound selected from the group consisting of
a compound represented by formula (B1): 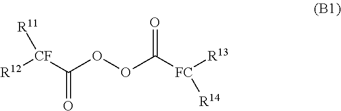
wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula
(B2): 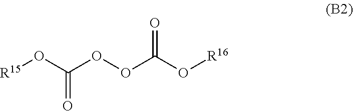
wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3): 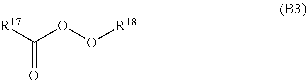
wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

14. The method for producing a fluorine-containing polymer (A) according to claim 10, wherein the decomposed product (C) of the polymerization initiator (B) is at least one compound selected from the group consisting of
a compound represented by formula (C1): 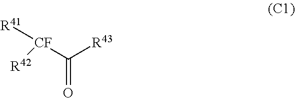
wherein R⁴¹ and R⁴² each independently represent a fluorine atom, a C1-C20 alkyl group, or a C1-C20 fluoroalkyl group,
the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula
(C2): 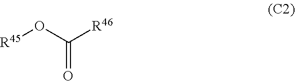
wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and
a compound represented by formula (C3): 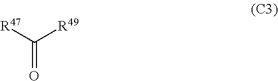
wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

15. The method for producing a fluorine-containing polymer (A) according to claim 10,
wherein the fluorine-containing monomer (M) is a compound represented by formula (M1): 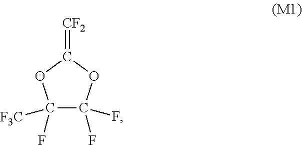
the structural unit represented by formula (A) is a structural unit represented by formula (A1):

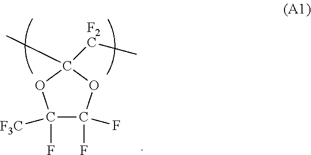

16. The method for producing a fluorine-containing polymer (A) according to claim 10,
wherein the fluorine-containing monomer (M) contains a compound (N) in an amount of 0.0001 to 10 mass % based on the mass of a starting monomer, and
the compound (N) is a compound represented by formula (N): 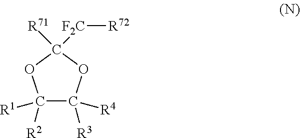
wherein R¹ to R⁴ each independently represent a fluorine atom, a C1-C5 perfluoroalkyl group, or a C1-C5 perfluoroalkoxy group, R⁷¹ and R⁷² each independently represent a hydrogen atom or a fluorine atom, and when one of R⁷¹ or R⁷² is a hydrogen atom, the other is a fluorine atom.

17. The method for producing a fluorine-containing polymer (A) according to claim 16, wherein the compound (N) is at least one compound selected from the group consisting of
a compound represented by formula (N1): 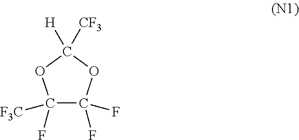 [SMILES: FC(F)(F)C1OC(F)(F)C(F)(C(F)(F)F)O1]
and
a compound represented by formula (N2):

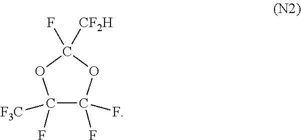 [SMILES: FC(F)C1(F)OC(F)(F)C(F)(C(F)(F)F)O1]

18. The method for producing a fluorine-containing polymer (A) according to claim 10, wherein the polymerization initiator (B) is at least one compound selected from the group consisting of a compound represented by formula (B1): 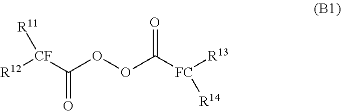
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5;
a compound represented by formula (B2): 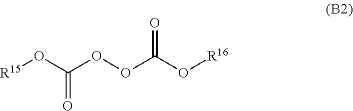
wherein R¹⁵ and R¹⁶ each independently represent a C1-C7 linear or branched alkyl group, or a C1-C7 linear or branched fluoroalkyl group; and
a compound represented by formula (B3): 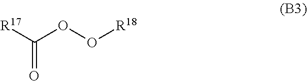
wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

19. The method for producing a fluorine-containing polymer (A) according to claim 10, wherein the decomposed product (C) is at least one compound selected from the group consisting of
a compound represented by formula (C1): 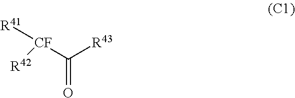
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2): 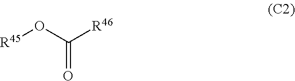
wherein R⁴⁵ represents a C1-C7 linear or branched alkyl group, or a C1-C7 linear or branched fluoroalkyl group, and R⁴⁶ represents a fluorine atom or a hydroxy group; and
a compound represented by formula (C3): 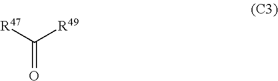
wherein R⁴⁷ represents methyl, tert-butyl, tert-nonyl, trifluoromethyl, perfluoroisopentyl, or phenyl, and R⁴⁹ represents a fluorine atom or a hydroxy group.

20. The method for producing a fluorine-containing polymer (A) according to claim 10, wherein the solvent (D) is at least one selected from the group consisting of hydrofluoroethers and fluorine-containing alcohols.

21. The method for producing a fluorine-containing polymer (A) according to claim 10,
wherein the fluorine-containing monomer (M) is a compound represented by formula (M1): 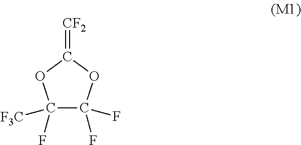
the fluorine-containing monomer (M) contains a compound (N) in an amount of 0.0001 to 10 mass % based on the mass of a starting monomer;
the compound (N) is at least one compound selected from the group consisting of a compound represented by formula (N1): 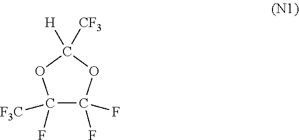 [SMILES: FC(F)(F)C1OC(F)(F)C(F)(C(F)(F)F)O1]
and
a compound represented by formula (N2), 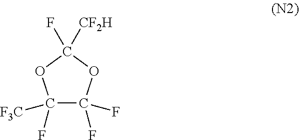 [SMILES: FC(F)C1(F)OC(F)(F)C(F)(C(F)(F)F)O1]
the structural unit represented by formula (A) is a structural unit represented by formula (A1): 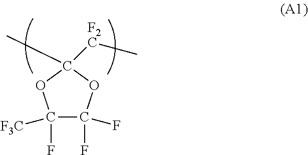
the polymerization initiator (B) is at least one compound selected from the group consisting of
a compound represented by formula (B1): 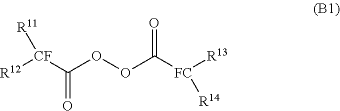
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula
(B2): 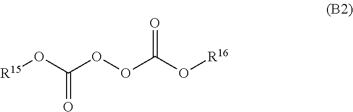
wherein R¹⁵ and R¹⁶ each independently represent a C1-C7 linear or branched alkyl group, or a C1-C7 linear or branched fluoroalkyl group, and
a compound represented by formula (B3): 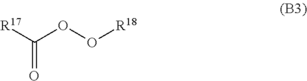
wherein R¹⁷ represents methyl, tert-butyl, tert-nonyl, trifluoromethyl, perfluoroisopentyl, or phenyl, and R⁸ represents tert-butyl, tert-hexyl, or trifluoromethyl;
the decomposed product (C) is at least one compound selected from the group consisting of
a compound represented by formula (C1): 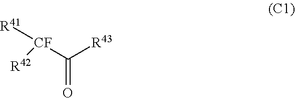
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2): 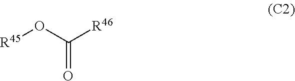
wherein R⁴⁵ represents a C1-C7 linear or branched alkyl group, or a C1-C7 linear or branched fluoroalkyl group, and R⁴⁶ represents a fluorine atom or a hydroxy group, and a compound represented by formula (C3): 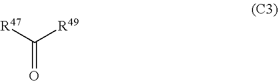
wherein R⁴⁷ represents methyl, tert-butyl, tert-nonyl, trifluoromethyl, perfluoroisopentyl, or phenyl, and R⁴⁹ represents a fluorine atom or a hydroxy group; and
the solvent (D) is at least one selected from the group consisting of hydrofluoroethers and fluorine-containing alcohols.

22. A lens for ultraviolet-light-emitting elements, the lens comprising a fluorine-containing polymer (A),
wherein the fluorine-containing polymer (A) comprises as a main component a structural unit represented by formula (A): 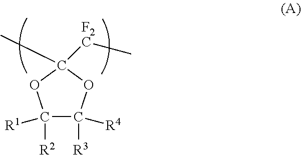
wherein R¹ to R⁴ each independently represent a fluorine atom, a C1-C5 perfluoroalkyl group, or a C1-C5 perfluoroalkoxy group, and
the fluorine-containing polymer (A) has an average transmittance of 85% or more in a wavelength range of 240 nm to 340 nm.

23. The lens for ultraviolet-light-emitting elements according to claim 22, wherein the structural unit represented by formula (A) is a structural unit represented by formula (A1):

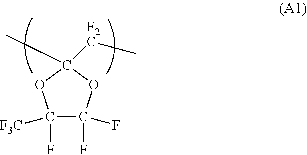

24. A composition comprising a fluorine-containing polymer (A1) produced as a precipitate by polymerizing a fluorine-containing monomer (M1) represented by formula (M1): 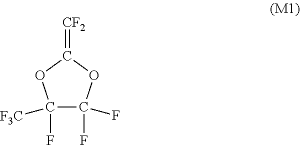
in the presence of the fluorine-containing monomer (M1), a polymerization initiator (B), and a solvent (D),
wherein the fluorine-containing polymer (A1) comprises as a main component a structural unit represented by formula (A1): 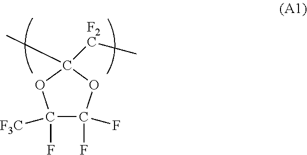
the fluorine-containing monomer (M1) contains at least one compound selected from the group consisting of
a compound represented by formula (N1): 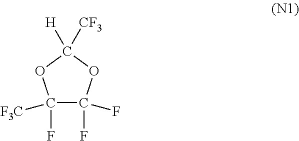 [SMILES: FC(F)(F)C1OC(F)(F)C(F)(C(F)(F)F)O1]
and
a compound represented by formula (N2): 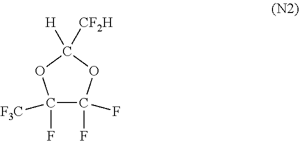 [SMILES: FC(F)C1OC(F)(F)C(F)(C(F)(F)F)O1]
in an amount of 0.0001 to 10 mass % based on the mass of a starting monomer;
a value (R_{A1}) of a solubility index R, calculated based on the Hansen solubility parameters of the solvent (D) and the Hansen solubility parameters of the fluorine-containing polymer (A1), is 4 or more;
a value (R_{B}) of a solubility index R, calculated based on the Hansen solubility parameters of the solvent (D) and the Hansen solubility parameters of the polymerization initiator (B), is 9 or less when the mass percentage of fluorine atoms in the polymerization initiator (B) is less than 50 mass %, and is 6 or less when the same mass percentage is 50 mass % or more;
a value (R_{C}) of a solubility index R, calculated based on the Hansen solubility parameters of the solvent (D) and the Hansen solubility parameters of the decomposed product (C) of the polymerization initiator (B), is 9 or less when the mass percentage of fluorine atoms in the decomposed product (C) is less than 50 mass %, and is 6 or less when the same mass percentage is 50 mass % or more; and
the solubility index R is calculated according to the following equation:
$$R = {\{ 4 \times {( δ {D}_{1} - δ {D}_{2} )}^{2} + {( δ {P}_{1} - δ {P}_{2} )}^{2} + {( δ {H}_{1} - δ {H}_{2} )}^{2} \}}^{0.5}$$
wherein δD₁, δP₁, and δH₁ respectively represent a dispersion parameter, a polarity parameter, and a hydrogen bonding parameter in the Hansen solubility parameters of element 1, and δD₂, δP₂, and δH₂ respectively represent a dispersion parameter, a polarity parameter, and a hydrogen bonding parameter in the Hansen solubility parameters of element 2.

25. The composition comprising a fluorine-containing polymer (A1) according to claim 24, wherein the polymerization initiator (B) is a peroxide.

26. The composition comprising a fluorine-containing polymer (A1) according to claim 24, wherein the polymerization initiator (B) is at least one compound selected from the group consisting of
a compound represented by formula
(B1): 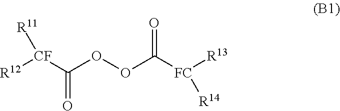
wherein R¹¹, R¹², R¹³, and R¹⁴ each independently represent a fluorine atom, a C1-C20 alkyl group, or a C1-C20 fluoroalkyl group,
the alkyl group and the fluoroalkyl group each optionally have an etheric oxygen atom,
R¹¹ and R¹² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R¹³ and R¹⁴ are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom;
a compound represented by formula
(B2): 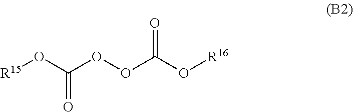
wherein R¹⁵ and R¹⁶ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom; and
a compound represented by formula (B3): 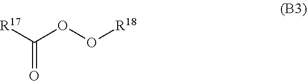
wherein R¹⁷ and R¹⁸ each independently represent a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and the alkyl group and the fluoroalkyl group each optionally have an etheric oxygen atom.

27. The composition comprising a fluorine-containing polymer (A1) according to claim 24, wherein the decomposed product (C) of the polymerization initiator (B) is at least one compound selected from the group consisting of
a compound represented by formula (C1): 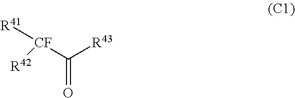
wherein R⁴¹ and R⁴² each independently represent a fluorine atom, a C1-C20 alkyl group, or a C1-C20 fluoroalkyl group,
the alkyl group and the fluoroalkyl group each optionally have an etheric oxygen atom, R⁴¹ and R⁴² are optionally linked together to form a 4- to 20-membered ring comprising 4 to 20 carbon atoms as ring-constituting atoms and at least one fluorine atom, or a 4- to 22-membered ring comprising 3 to 20 carbon atoms and one or two oxygen atoms or one sulfur atom as ring-constituting atoms and at least one fluorine atom, and
R⁴³ represents a fluorine atom or a hydroxy group;
a compound represented by formula
(C2): 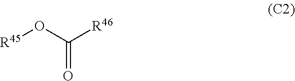
wherein R⁴⁵ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, and R⁴⁶ represents a fluorine atom or a hydroxy group; and
a compound represented by formula (C3): 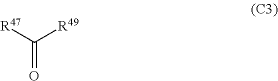
wherein R⁴⁷ represents a C1-C20 linear or branched alkyl group, a C1-C20 linear or branched fluoroalkyl group, a C4-C8 cyclic alkyl group, a C4-C8 cyclic fluoroalkyl group, or a C4-C14 aryl group optionally having a fluorine atom, the alkyl group and the fluoroalkyl group each optionally have an etheric oxygen atom, and R⁴⁹ represents a fluorine atom or a hydroxy group.

28. The composition comprising a fluorine-containing polymer (A1) according to claim 24, wherein the polymerization initiator (B) is at least one compound selected from the group consisting of
a compound represented by formula (B1): 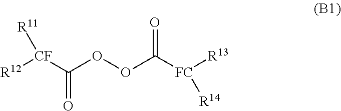
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5;
a compound represented by formula (B2): 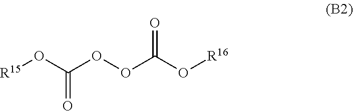
wherein R¹⁵ and R¹⁶ each independently represent a C1-C7 linear or branched alkyl group, or a C1-C7 linear or branched fluoroalkyl group; and
a compound represented by formula (B3): 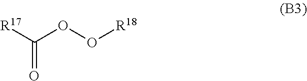
wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl.

29. The composition comprising a fluorine-containing polymer (A1) according to claim 24, wherein the decomposed product (C) is at least one compound selected from the group consisting of
a compound represented by formula (C1): 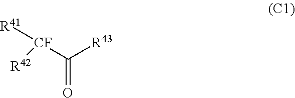
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group;
a compound represented by formula (C2): 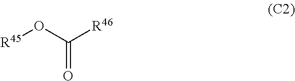
wherein R⁴⁵ represents a C1-C7 linear or branched alkyl group, or a C1-C7 linear or branched fluoroalkyl group, and R⁴⁶ represents a fluorine atom or a hydroxy group; and
a compound represented by formula (C3): 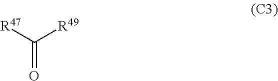
wherein R⁴⁷ represents methyl, tert-butyl, tert-nonyl, trifluoromethyl, perfluoroisopentyl, or phenyl, and R⁴⁹ represents a fluorine atom or a hydroxy group.

30. The composition comprising a fluorine-containing polymer (A1) according to claim 24, wherein the solvent (D) is at least one selected from the group consisting of hydrofluoroethers and fluorine-containing alcohols.

31. The composition comprising a fluorine-containing polymer (A1) according to claim 24,
wherein the polymerization initiator (B) is at least one compound selected from
the group consisting of
a compound represented by formula (B1): 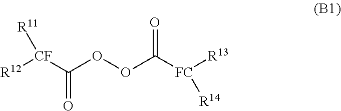
wherein R¹¹ and R¹³ each represent a fluorine atom, and
R¹² and R¹⁴ each independently represent
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5,
a compound represented by formula
(B2): 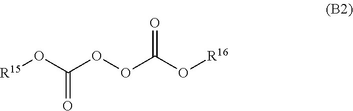
wherein R¹⁵ and R¹⁶ each independently represent a C1-C7 linear or branched alkyl group, or a C1-C7 linear or branched fluoroalkyl group, and
a compound represented by formula (B3): 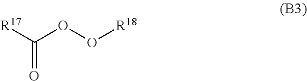
wherein R¹⁷ represents methyl, tert-butyl, tert-nonyl, trifluoromethyl, perfluoroisopentyl, or phenyl, and R¹⁸ represents tert-butyl, tert-hexyl, or trifluoromethyl;
the decomposed product (C) is at least one compound selected from the group consisting of
a compound represented by formula (C1): 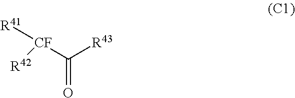
wherein R⁴¹ represents a fluorine atom,
R⁴² represents
—(CF₂)ₙ₁CF₃ wherein n1 represents an integer of 0 to 5,
—(CF₂)ₙ₂CF₂H wherein n2 represents an integer of 0 to 5,
—O—(CF₂)ₙ₃CF₃ wherein n3 represents an integer of 0 to 5, or
—O—(CF₂)ₙ₄CF₂H wherein n4 represents an integer of 0 to 5, and
R⁴³ represents a fluorine atom or a hydroxy group,
a compound represented by formula (C2): 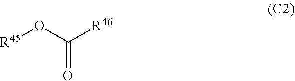
wherein R⁴⁵ represents a C1-C7 linear or branched alkyl group, or a C1-C7 linear or branched fluoroalkyl group, and R⁴⁶ represents a fluorine atom or a hydroxy group, and
a compound represented by formula (C3): 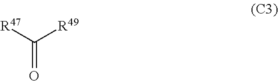
wherein R⁴⁷ represents methyl, tert-butyl, tert-nonyl, trifluoromethyl, perfluoroisopentyl, or phenyl, and R⁴⁹ represents a fluorine atom or a hydroxy group; and
the solvent (D) is at least one selected from the group consisting of hydrofluoroethers and fluorine-containing alcohols.
